SEQUENCE LISTING (2 sequences)

SEQ ID NO: 1 - DNA, 20 bp, Phytophthora infestans
ctcgctacaataggagggtc

SEQ ID NO: 2 - DNA, 18 bp, Phytophthora infestans
cggaccgcctgcgagtcc

(19) United States
(12) Patent Application Publication
(10) Pub. No.: US 2022/0236242 A1
(43) Pub. Date: Jul. 28, 2022

(54) METHODS AND SYSTEMS FOR ASSESSING PLANT CONDITIONS BY VOLATILE DETECTION

(71) Applicant: North Carolina State University, Raleigh, NC (US)

(72) Inventors: Quingshan Wei, Raleigh, NC (US); Zheng Li, Raleigh, NC (US); Jean Beagle Ristaino, Raleigh, NC (US)

(21) Appl. No.: 17/626,644

(22) PCT Filed: Jul. 13, 2020
(86) PCT No.: PCT/US2020/041764

§ 371 Date: Jan. 12, 2022

Related U.S. Application Data
(60) Provisional application No. 62/873,561, filed on Jul. 12, 2019.

(51) Int. Cl. G01N 31/22; G01N 33/00

(52) U.S. Cl. CPC G01N 31/22; G01N 33/0098

(57) ABSTRACT

Methods and systems for assessing plant conditions by volatile detection. The subject matter of the present disclosure describes a cost-effective, compact, noninvasive volatile organic compound (VOC) fingerprinting platform installed on a consumer electronics device such as a smartphone, tablet, or handheld device, or other mobile device for the early detection and/or diagnosis of disease in a plant caused by infection by a plant pathogen such as Altemaria solani, Septoria lycopersici, or Phytophthora infestans, based on the pattern analysis of characteristic leaf volatile emissions. This handheld device integrates a sensor array to be imaged by the smartphone camera and a micropump for active sampling and real-time detection.

### CROSS-REFERENCE TO RELATED APPLICATIONS

This application claims benefit of U.S. Provisional Patent Application Ser. No. 62/873,561, filed Jul. 12, 2019, the entire disclosure of which is expressly incorporated by reference herein.

### TECHNICAL FIELD

The presently disclosed subject matter relates to systems and related methods that can be used to detect the presence and/or amount of volatile organic compound(s) (VOC) in a plant sample, for example, to determine the presence of disease or other stress condition in the plant.

### BACKGROUND

Plant diseases cause severe threats to global food security by devastating crop production in every region of the world. Statistically, around 20-40% of all crops losses globally are due to pre- or post-harvest plant diseases¹; in the U.S., estimated annual crop losses due to non-indigenous arthropod species and plant pathogen introductions are $14.1 and $21.5 billion, respectively. Late blight caused by Phytophthora infestans (Mont.) de Bary^{3,4} is one of the most “armed and dangerous” plant diseases⁵ with serious implications on the production of economically important crops such as potato and tomato. Late blight alone accounts for global financial losses of nearly five billion dollars⁶. Late blight is identified by blackish-brown lesions on the surface of plant tissues that result in sporulation of P. infestans, spread of sporangia to other plants, and death of infected plants in a few days if the plants are left untreated. Furthermore, the pathogen spreads rapidly under favorable weather conditions. In the 2009 late blight pandemic in the eastern U.S., it only took about 2 weeks for the pathogen to spread from infected transplants to over 50% of the counties in New York⁷. Therefore, developing a rapid and effective method for early diagnosis of P. infestans and many other plant pathogens is critical to the prevention of spread of pathogens and subsequent crop diseases and reduction of economic losses in agriculture.

Currently, plant pathogen detection is heavily focused on a wide variety of molecular assays, including nucleic acid-based technologies such as polymerase chain reaction (PCR)^{8, 9}, loop-mediated isothermal amplification (LAMP)^{10, 11}, or DNA microarrays¹², and immunological approaches such as antibody-based lateral flow assays (LFA)¹³ and enzyme-linked immunosorbent assays (ELISA)^{14, 15}. Nucleic acid-based methods are sensitive and specific, but dependent on cumbersome assay protocols. Immunoassay technology on the other side offers simplicity and portability for on-site detection, but is limited by detection sensitivity and specificity for certain applications. Alternatively, field-portable sensors have seen rapid development in the past few years and hold great promise. For example, a few lab-on-a-chip PCR devices for detection of plant pathogens have recently been demonstrated^{16, 17, 18}. However, few miniature systems are capable of high analytical performance while at the same time maintaining simplicity and cost-effectiveness.

### SUMMARY

In accordance with this disclosure systems, devices, and methods for assessing plant health conditions by volatile detection are provided. In one aspect, a system for detecting a presence and/or an amount of one or more volatile organic compounds (VOC) in a plant sample, the system comprising: a receiver configured to receive a gaseous emission from a plant sample, the receiver comprising one or more sensing elements, each sensing element comprising one or more sensors, that react with one or more VOC in the gaseous emission; and a detector comprising one or more cameras, the detector being configured for detecting a signal associated with the reacting of the one or more sensing elements with one or more VOC in the gaseous emission.

In some embodiments, the one or more sensing elements comprise a nanosensor, a dye, or a combination thereof. In some embodiments, the nanosensor is functionalized with a ligand that reacts with the one or more VOC in the gaseous emission. In some embodiments, wherein the nanosensor is shape-controlled, optionally wherein the nanosensor comprises a nanoparticle, a nanorod, or other shapes. In some embodiments, the nanosensor has a dimension ranging from between, and including, about 5 and 200 nanometers (nm) and/or an aspect ratio of about 1 to about 6. In some embodiments, the nanosensor comprises gold, silver, copper, aluminum, or any alloy thereof. In some embodiments, the nanosensor, optionally the shape-controlled nanosensor, has an absorption range of between, and including, about 400 and 1200 nm, optionally wherein the nanosensor comprises a nanoparticle having an absorption range of between, and including, about 520 and 580 nm and/or wherein the nanosensor comprises a nanorod having a longitudinal resonance in the range of between, and including, about 530 and 1000 nm.

In some embodiments, the system comprises between, and including, about 2 and 100 sensing elements, optionally, between, and including, about 2 and 10 sensing elements, further optionally wherein the sensing elements of the receiver are configured in a linear microarray. In some embodiments, the one or more cameras of the detector is configured to capture one or more images of the receiver. In some embodiments, the detector comprises a consumer electronics device comprising a light source configured to illuminate the receiver, optionally wherein the consumer electronics device is a smart phone, a tablet, or other mobile device. In some embodiments, the detector comprises an attachment configured to position the receiver with respect to the camera and/or light source, optionally wherein the attachment further comprises a lens, a diffuser or a combination thereof. In some embodiments, the system further comprises a pump configured to direct the gaseous emission to the receiver.

In some embodiments, the system further comprises one or more processors configured to determine the presence and/or the amount the one or more VOC in the plant sample. In some embodiments, the receiver comprises a material selected from the group consisting of paper and a hydrophobic nanoporous substrate; and optionally, wherein the hydrophobic nanoporous substrate is selected from the group consisting of a silica sol-gel, a polymer membrane, and a metal organic framework (MOF).

In another aspect, a nanosensor is provided that reacts with one or more VOC in a gaseous emission from a plant sample. In some embodiments, the nanosensor is functionalized with a ligand that reacts with the one or more VOC in the gaseous emission. In some embodiments, the nanosensor is shape-controlled, optionally wherein the nanosensor comprises a nanoparticle or a nanorod. In some embodiments, the nanosensor has a dimension ranging between, and including, about 5 and 200 nanometers (nm) and/or an aspect ratio of between, and including, about 1 to 6. In some embodiments, the nanosensor comprises gold, silver, copper, aluminum, or any alloy thereof. In some embodiments, the nanosensor, optionally the shape-controlled nanosensor, has an absorption range of between, and including, about 400 and 1200 nm, optionally wherein the nanosensor comprises a nanoparticle having an absorption range of between, and including, about 520 and 580 nm, and/or wherein the nanosensor comprises a nanorod having a longitudinal resonance in the range of between, and including, about 530 and 1000 nm.

In yet another aspect, a method for detecting a presence and/or an amount of one or more volatile organic compounds (VOC) in a plant sample, the method comprising: providing a plant sample; exposing a gaseous emission from the plant sample to one or more sensing elements, each sensing element comprising one or more sensors, that react with the one or more VOC in the gaseous emission; detecting a signal associated with the reacting of the one or more VOC with the one or more sensing elements; and detecting the presence and/or the amount of the one or more VOC based on the signal.

In some embodiments, the one or more sensing elements comprise a nanosensor, a dye, or a combination thereof. In some embodiments, the nanosensor is functionalized with a ligand that reacts with the one or more VOC in the gaseous emission. In some embodiments, the nanosensor is shape-controlled, optionally wherein the nanosensor comprises a nanoparticle or a nanorod. In some embodiments, the nanosensor has a dimension ranging between, and including, about 5 and 200 nanometers (nm) and/or an aspect ratio of between, and including, about 1 and 6. In some embodiments, the nanosensor comprises gold, silver, copper, aluminum, or any alloy thereof. In some embodiments, the nanosensor, optionally the shape-controlled nanosensor, has an absorption range of between, and including, about 400 and 1200 nm, optionally wherein the nanosensor comprises a nanoparticle having an absorption range of between, and including, about 520 and 580 nm and/or wherein the nanosensor comprises a nanorod having a longitudinal resonance in the range of between, and including, about 530 and 1000 nm. In some embodiments, the one or more sensing elements are configured on a receiver, optionally wherein the receiver comprises a material selected from the group consisting of paper and a hydrophobic nanoporous substrate, further optionally wherein the hydrophobic nanoporous substrate is selected from the group consisting of a silica sol-gel, a polymer membrane, and a metal organic framework (MOF).

In some further embodiments, the method further comprises between, and including, about 2 and 100 sensing elements, optionally between, and including, about 2 and 10 sensing elements, further optionally wherein the sensing elements of the receiver are configured in a linear microarray. In some embodiments, the method further comprises detecting a signal comprises capturing one or more images of the one or more sensing elements with a camera. In some embodiments, detecting a signal comprises using a consumer electronics device having a camera configured to capture one or more images of the receiver and a light source configured to illuminate the receiver, optionally wherein the consumer electronics device is a smart phone, a tablet, or some other mobile device. In some embodiments, detecting a signal comprises employing an attachment configured to position the one or more sensing elements with respect to the camera and/or light source, optionally wherein the attachment further comprises a lens, a diffuser, or a combination thereof.

In some further embodiments, the method further comprises directing the gaseous emission to the one or more sensing elements using a pump. In some embodiments, the plant sample is a field sample or a sample from a plant product. In some embodiments, the method further comprising determining a condition of the plant or plant product based on the presence and/or the amount of the one or more VOC. In some embodiments, the condition of the plant is an infection, an asymptomatic infection, a contamination by a foodborne microorganism, an abiotic stress condition, a pest infestation, or a combination thereof. In some embodiments, the infection is an infection caused by a fungus, bacterium, virus, oomycete, other plant pathogen, or insect pest. In some embodiments, the method further comprises generating a profile of one or more signals from the one or more sensing elements based on the condition of the plant. In some embodiments, generating a profile comprises generating a profile to identify and/or distinguish individual species of organism. In another aspect, a profile generated by the methods described above are provided.

Accordingly, it is an object of the presently disclosed subject matter to provide systems and related methods that can be used to detect the presence and/or amount of volatile organic compound(s) (VOC) in a plant sample, for example, to determine the presence of disease, contamination, pest infestation or other stress condition in the plant. This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description of the presently disclosed subject matter and non-limiting Examples and Figures.

### BRIEF DESCRIPTION OF THE FIGURES

The features and advantages of the present subject matter will be more readily understood from the following detailed description which should be read in conjunction with the accompanying drawings that are given merely by way of explanatory and non-limiting example, and in which:

FIG. 1 illustrates a bottom view of a volatile organic compound (VOC) sensing system according to some embodiments of the present disclosure;

FIG. 2A, FIG. 2B, and FIG. 2C illustrate several views of a case for a mobile device, the case being retrofitted with a sensor holder for the VOC sensing system according to some embodiments of the present disclosure;

FIG. 3 illustrates an exploded view of several components of the VOC sensing system according to some embodiments of the present disclosure;

FIG. 4A illustrates a top view of a sensor cartridge of the VOC sensing system according to some embodiments of the present disclosure;

FIG. 4B illustrates an exploded view of the sensor cartridge of the VOC sensing system according to some embodiments of the present disclosure;

FIG. 4C illustrates a sensor array of the sensor cartridge and includes details on how gas flows through the sensor array of the VOC sensing system according to some embodiments of the present disclosure;

FIG. 5 illustrates an alternative embodiment of a VOC sensing system where the sensor array is not attached to a mobile device, according to some embodiments of the present disclosure;

FIG. 6 illustrates a top view of the mobile device of the VOC sensing system according to some embodiments of the present disclosure;

FIG. 7 illustrates a bottom view of the mobile device, including the sensor cartridge and a diaphragm micropump attached, of the VOC sensing system according to some embodiments of the present disclosure;

FIG. 8 illustrates a schematic of the aggregation of gold nanorods occurring at the gas-solid interface induced by exposure to (E)-2-hexenal;

FIG. 9 illustrates the sensor response of a multiplex array to plant volatiles for 1-minute exposure and their chemometric analysis;

FIG. 10 illustrates sensor response matrices before and after exposure to the gases and a difference map illustrating the major color differences between the “Before Exposure” matrix and the “After Exposure” matrix;

FIG. 11 illustrates RGB differential sensor response profiles of 10 representative plant volatiles at 10 ppm after the nanosensors and dyes have been exposed to the plant volatiles;

FIG. 12A illustrates differential RGB differential sensor response profiles of a healthy control compared to sensor exposure to volatiles released from infected tomato leaves up to six days after inoculation with P. infestans;

FIG. 12B illustrates a response plot showing the Euclidean distance (ED) of all 10 sensor elements as a function of the duration of pathogen infection;

FIG. 13A illustrates differential RGB sensor response profiles of a healthy control compared to sensor exposure to 3 different plant pathogens in an inoculated tomato leaf; and

FIG. 13B illustrates a PCA plot of infected tomato leaves versus the healthy control.

### DETAILED DESCRIPTION

The present disclosure provides, in some embodiments, a cost-effective, compact, noninvasive volatile organic compound (VOC) fingerprinting platform installed on a consumer electronics device such as a smartphone, tablet, web cam, drone, or other handheld or mobile device for the early detection and/or diagnosis of disease in a plant caused by infection by a plant pathogen such as Alternaria solani, Septoria lycopersici, or Phytophthora infestans, based on the pattern analysis of characteristic leaf volatile emissions. This handheld device integrates a sensor array to be imaged by the smartphone camera or a camera from a mobile digital device and a micropump for active sampling and real-time detection or near real-time detection.

Thus, provided in accordance with some embodiments of the presently disclosed subject matter is a cost-effective, compact, and noninvasive volatile organic compound (VOC) fingerprinting platform installed on a consumer electronics device such as a smartphone for the early detection and/or diagnosis of disease in a plant caused by infection by a plant pathogen such as Phytophthora infestans, Alternaria solani or, Septoria lycopersici, based on the pattern analysis of characteristic leaf volatile emissions. This handheld device integrates a sensor array to be imaged by the smartphone camera and a micropump for active sampling and real-time detection or near real-time detection, for example and without limitation, any images captured by the camera, smartphone, or mobile device camera can be uploaded immediately after being captured (e.g., such as via the Internet, a cloud-based system, or any other wireless system) to a computing system (e.g., one or more processors) for analysis or the handheld device itself can have an computer application (e.g., a smartphone mobile application) configured to perform the image analysis process. Although the images can be uploaded at near-real time after being captured, some time is required for analysis of the images as described herein. Alternatively, once the camera has captured one or more images, the image data can be manually transferred to a computing platform via a memory stick, USB drive, SD card, or any other suitable medium. In some embodiments, a multiplexed paper-based chemical sensor array comprises 10 sensor elements that incorporate functionalized gold nanomaterials and chemo-responsive organic dyes to detect key plant volatiles (e.g. green leaf volatiles (GLVs), phytohormones, etc.) at parts per million (ppm) level detection limit within a one minute reaction. Combined with a statistical method such as principal component analysis (PCA) in some embodiments, the presently disclosed system provided for simultaneous detection and classification of 10 individual plant volatiles, including two characteristic late blight VOC markers (E-2-hexenal and 2-phenylethanol). This allowed early detection of tomato late blight caused by Phytophthora infestans 2 days after inoculation in asymptomatic plants, and differentiation from other fungal pathogens that lead to similar symptoms of tomato foliage (i.e., Alternaria solani, cause of early blight and Septoria lycopersici, cause of Septoria leaf spot). Given the flexibility of sensor design and cost-effectiveness, a smartphone-based VOC sensing system in accordance with the presently disclosed subject matter can be broadly applied to monitoring various other important plant diseases, foodborne contamination, or abiotic stresses through the rapid profiling of characteristic volatile emissions.

The presently disclosed subject matter will now be described more fully hereinafter with reference to the accompanying Figures and Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art. Certain components in the figures are not necessarily to scale, emphasis instead being placed upon illustrating the principles of the presently disclosed subject matter (in some cases schematically).

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

While the following terms are believed to be well understood by one of ordinary skill in the art, the following definitions are set forth to facilitate explanation of the presently claimed subject matter.

Following long-standing patent law convention, the terms “a”, “an”, and “the” refer to “one or more” when used herein, including in the claims.

As used herein, the term “about”, when referring to a value or an amount, for example, relative to another measure, is meant to encompass variations of in some embodiments ±20%, in some embodiments ±10%, in some embodiments ±5%, in some embodiments ±1%, and in some embodiments ±0.1% from the specified value or amount, as such variations are appropriate. The term “about” can be applied to all values set forth herein.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed in some embodiments as a “p-value”. Those p-values that fall below a user-defined cutoff point are regarded as significant. In some embodiments, a p-value less than or equal to 0.05, in some embodiments less than 0.01, in some embodiments less than 0.005, and in some embodiments less than 0.001, are regarded as significant.

Referring to FIG. 1, in some embodiments, the presently disclosed subject matter provides a system 100 for detecting a presence and/or an amount of one or more volatile organic compounds (VOC) in a plant sample. In some embodiments, the system 100 comprises a mobile device (not visible in FIG. 1) with a receiver 106 attached to the mobile device case 102 by a receiver holder 104. In some embodiments, the receiver 106 is configured to receive a gaseous emission from a plant sample, for example the plant sample in the vial V, the receiver 106 comprising a substrate and one or more sensing elements that react with one or more VOC in the gaseous emission, and a detector (not visible in this view) configured for detecting a signal (i.e., a color change, fluorescent signal, electric signal, change in appearance, or other appropriate visual indication) associated with the reacting of the one or more sensing elements with one or more VOC in the gaseous emission.

In the systems and methods of the presently disclosed subject matter, directing the gaseous emission to the one or more sensing elements can be accomplished by any suitable approach or device as would be apparent to one of ordinary skill in the art upon a review of the instant disclosure. For example and without limitation, in some embodiments, the system 100 comprises a diaphragm micropump chamber 110 configured to direct the gaseous emission to the receiver 106. As shown by the directional arrows 108 gaseous emissions flow from the vial V into, through, and out the receiver 106 and into the diaphragm micropump chamber 110.

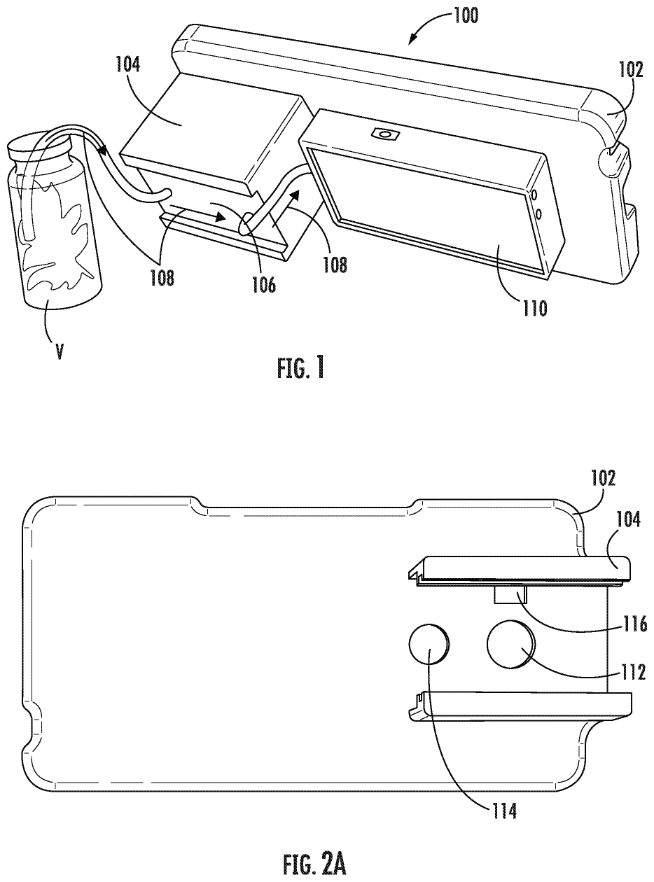

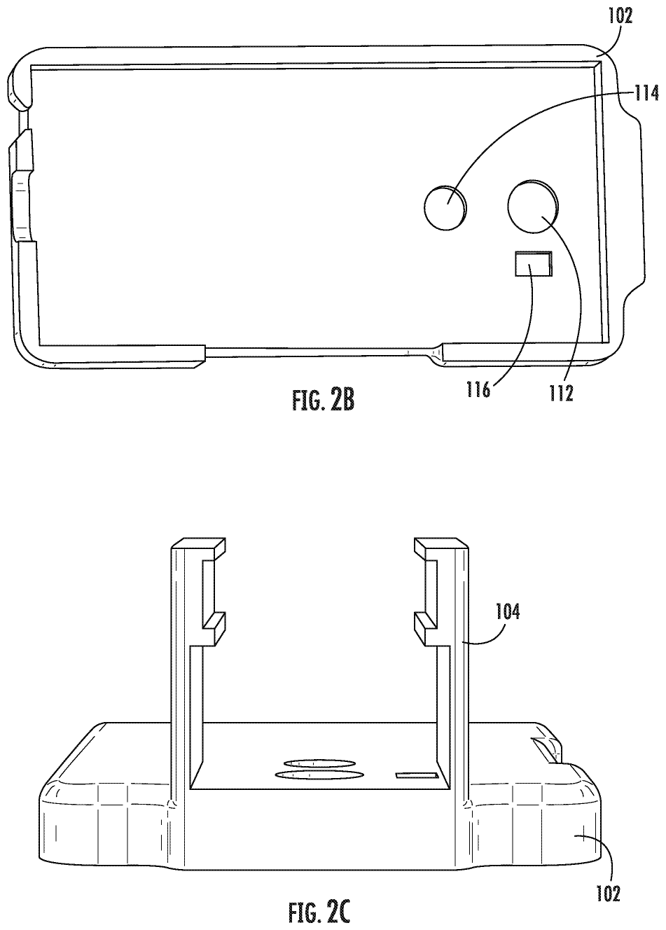

Referring to FIG. 2A through FIG. 2C, which illustrates different views of a mobile device case 102 used to facilitate and integrate certain aspects of the system 100. For example and without limitation, the mobile device case 102 comprises a holder 104 for the receiver 106 and the diaphragm micropump chamber 110 can be connected to the mobile device case 102 as well. As the name implies, the mobile device case 102 can also be configured to have a mobile device, such as, for example and without limitation, a smartphone, tablet, personal data assistant (PDA), or other suitable mobile device attached to it. In addition to the receiver holder 104, the mobile device case 102 comprises a camera hole 112, an unlock button 114, and a light hole 116. In some embodiments, the camera hole 112 is configured to be positioned at or approximately at the location of the camera of the mobile device. For example and without limitation, the camera hole 112 can be positioned such that the camera of the mobile device can completely view through the camera hole 112 without obstruction from the mobile device case 102. In some embodiments, the camera hole 112 can be used to hold a piece of an external lens, if needed. The external lens (described further herein, can help adjust the field of view and spatial resolution. In addition, the camera hole 112 can hold an optical emission filter, if fluorescent test strip is used and fluorescent detection is needed. In some further embodiments, the light hole 116 can be positioned and configured such that light from the LED or other light from the mobile device can shine through the light hole 116 without obstruction.

Referring to FIG. 3, an exploded view of the system 100 is illustrated, this time including a mobile device S. In some embodiments of the system 100, the mobile device S can be inserted into the mobile device case 102 where the various holes described above align with the camera and light of the mobile device S. For example and without limitation, as the mobile device S is inserted into the mobile device case 102, the camera 118 of the mobile device S can be positioned to capture images through the camera hole (not visible in this view). As described above, as the gaseous emission flows through the receiver 106, the gaseous emission comes into contact with the sensing element 106-1 that react with the gaseous emission. As the sensing element 106-1 reacts with the gaseous emission, in some embodiments, the camera 118 is configured to capture one or more images of the sensing element 106-1. To help capture images the entire sensing element 106-1, an external lens 120 can be provided to achieve a greater viewing angle 122 of the sensing element 106-1. In some embodiments, the external lens 120 can be about 12 mm in diameter with a focal distance of about 48 mm. In some embodiments, the external lens 120 can provide a demagnification factor of about 6 times, with a 30 mm distance from the receiver 106, such that the entire sensing element 106-1 can be captured in the field of view of the camera 118.

Continuing with reference to FIG. 3, in some embodiments, the mobile device S comprises a light 124 which can be a flashlight or a camera flash or any other suitable light. In some embodiments, the light 124 is configured to shine through the mobile device case 102, without interference, to shine on the sensing element 106-1 to properly light the sensing element 106-1 for the camera 118 to capture one or more images of the sensing element 106-1. In some embodiments, the system 100 can comprise an optical diffuser 126 configured to ensure that the illumination 128 provided by the light 124 is uniform.

Those having ordinary skill in the art will appreciate that the design illustrated for the mobile device S and the mobile device case 102, can be changed, altered, or reconfigured to work for any mobile device. For example and without limitation, the components described herein can be modified or altered to accommodate any smartphone, tablet, or other mobile device from Android, Apple, Microsoft, Samsung, etc. In such a reconfiguration, the dimensions of the mobile device case 102 would change as well as the placement and sizes of the holes and potentially the characteristics of the external lens 120. In any event, the goal is to provide a system 100 that has the ability to receive the gaseous emissions, capture images of the sensing element 106-1 as it is exposed to the gaseous emissions, and light the sensing element 106-1 adequately to capture said images.

In some embodiments, the receiver 106 is configured as a cartridge, meaning it can easily be installed and removed from the receiver holder 104. This is so the receiver 106 can be exchanged for other receivers of the same or different type.

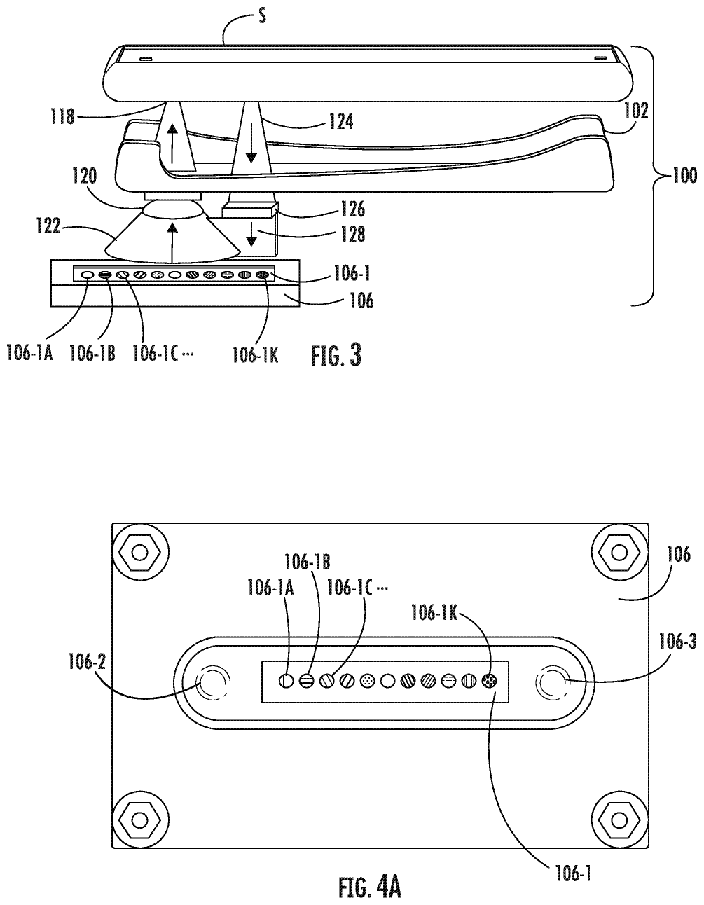

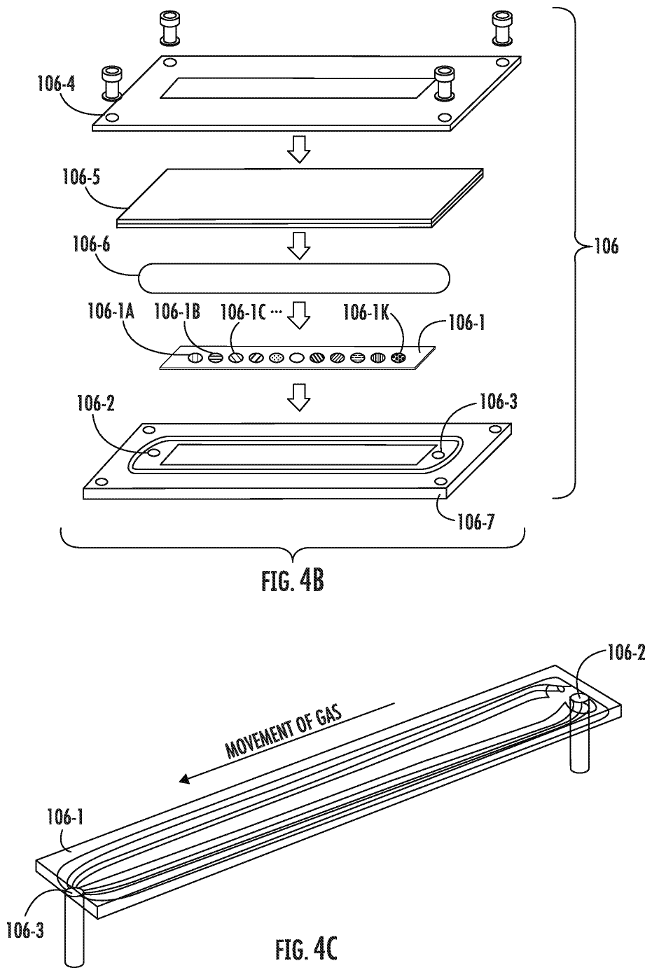

Referring to FIG. 4A, which is a close-up illustration of a camera facing view of the receiver 106, in some embodiments, the receiver 106 comprises an inlet 106-2, where gaseous emissions can enter the receiver 106 from a plant sample, such as the one shown in FIG. 1, and then enter the sensing element 106-1. Additionally, in some embodiments, the receiver 106 comprises an outlet 106-3, where a pump can connect to the receiver 106 to draw gaseous emissions through the sensing element 106-1 and the receiver 106. In some embodiments, the receiver 106 is alternatively referred to as a solid support, as a solid support is an example of a suitable receiver. As described in the specification of FIG. 3, the sensing element 106-1 faces the camera 118 of the mobile device S such that the camera 118 can capture a picture of the sensing element 106-1.

Referring to FIG. 4B, in some embodiments, the receiver 106 comprises the sensing element 106-1, which comprises a substrate (e.g., the rectangular sheet that the circles are placed on). In some embodiments, the substrate can comprise a paper strip, such as, for example and without limitation, a nitrocellulose paper substrate. By way of additional example and not limitation, the sensing element 106-1 can comprise any hydrophobic nanoporous substrate, such as a silica sol-gel, a polymer membrane, and/or a metal-organic framework (MOF).

In some embodiments, the substrate of the one or more sensing elements 106-1 can comprise one or more individual sensors 106-1A through 106-1K, such as, for example and without limitation, a nanosensor, a dye, or a combination thereof (i.e., the circles on the rectangular sheet of the sensing element 106-1) arranged on the paper substrate. In some embodiments, the one or more sensing elements 106-1 can comprise a plurality of nanosensors, dyes, or combination thereof, each of the nanosensors or dyes arranged on the paper substrate. In some embodiments, the one or more individual sensors 106-1A through 106-1K can be arranged on the paper substrate in any suitable manner. In some other embodiments, the one or more individual sensors 106-1A through 106-1K can be arranged in an array to create a VOC sensor array. In some embodiments, the presently disclosed subject matter provides a colorimetric VOC sensor array. In some embodiments, fluorescent nanomaterials/dyes are used to form a fluorescent VOC sensor array. In some embodiments, the nanosensor is functionalized with one or more ligand that reacts with the one or more VOC in the gaseous emission. The nanomaterials/dyes can provide a colorimetric and/or fluorescent signal. For example and without limitation, the one or more ligand can comprise cysteine (Cys), phenols, thiourea, cavitand molecules, etc. Some example plant VOCs that can be tested by the system 100 of the present disclosure include but are not limited to: E-2-Hexenal, Z-3-Hexenal, 1-Hexanal, E-2-Hexenol, Benzaldehyde, 4-Ethylguaiacol, 4-Ethyphenol, Methyl Jasmonate, Methyl Salicylate, 2-Phenylethanol.

In some embodiments, the nanosensor(s) comprises a nanoparticle, a nanorod, and/or other shapes. Thus, in some embodiments, the nanosensor is “shape-controlled.” By way of additional example and not limitation, other shapes include cubes, prisms, discs, and the like. In some embodiments, the nanosensor has a dimension ranging between, and including, about 5 nanometers (nm) and 200 nm, including about 5, 10, 15, 20, 25, 30, 35, 40, 45, 50, 55, 60, 65, 70, 75, 80, 85, 90, 95,100, 105, 110, 115, 120, 125, 130, 135, 140, 145, 150, 155, 160, 175, 180, 185, 190, 195, 200 nm, and another other appropriate size in between any value listed above. In some embodiments, the nanosensor has an aspect ratio ranging from about 1 to about 6, including aspect ratios of 1, 2, 3, 4, 5, or 6, and fractional values therebetween, e.g. 2.5, 3.4, and the like. By way of particular example and not limitation, the nanosensor comprises a nanoparticle that has a dimension ranging between, and including, about 5 and 200 nanometers (nm). In some embodiments, the nanosensor comprises a nanorod that has an aspect ratio of between, and including, about 1 and 6.

In some embodiments the nanosensor comprises a material selected from the group comprising gold, silver, copper, aluminum, or an alloy thereof. By way of particular example, the nanosensor comprises a nanoparticle and/or nanorod that comprises gold.

Depending on the combination of metal materials, size, and shape, the absorption can be tuned from the visible to near infrared. In some embodiments, the nanosensor, in some embodiments a shape-controlled nanosensor, has an absorption wavelength range of between, and including, about 400 and 1200 nm, including 450, 500, 520, 530, 550, 580, 600, 650, 700, 750, 800, 850, 900, 930, 950, 1000, 1050, 1100, 1150, or 1200 nm, and any value between these ranges. By way of particular example and not limitation, a nanosensor comprises a nanoparticle having an absorption wavelength range of between, and including, about 520 and 580 nm and/or the nanosensor comprises a nanorod that has a longitudinal resonance in the range of between, and including, about 530 and 1000 nm.

In the systems and methods of the presently disclosed subject matter, any desired number of individual sensors 106-1A through 106-1K can be included, depending for example on the condition to be assessed and/or profile to be determined and/or cost targets for the system 100. As a general suggestion but not a requirement, more individual sensors 106-1A through 106-1K can provide better performance. In some embodiments, 2 to 100 individual sensors 106-1A through 106-1K are employed, including 2, 5, 10, 20, 30, 40, 50, 50, 70, 80, 90, 100, or any value in between, individual sensors 106-1A through 106-1K. The individual sensors 106-1A through 106-1K can be deployed in any suitable configuration as would be apparent to one of ordinary skill in the art upon a review of the instant disclosure. For example, the configuration can be based on the mobile device (i.e., detector) used. By way of particular example and not limitation, the individual sensors 106-1A through 106-1K are configured in a linear microarray.

As illustrated in FIG. 4B, in some embodiments each receiver cartridge 106 comprises several layers, including a top layer 106-4 and a bottom layer 106-7. The top layer 106-4 has an open window for the camera to image the sensing element 106-1. The bottom layer 106-7 comprises the inlet 106-2 and outlet 106-3 where the gaseous emissions are configured to enter and exit, respectively, the receiver cartridge 106. In some embodiments, the receiver cartridge 106 further comprises a transparent layer 106-5 to hold the sensing element 106-1 in place. For example, and without limitation, in some embodiments, the transparent layer 106-5 can be a microscope cover glass or any other suitable device used to hold down the sensing element 106-1 for imaging. The transparent layer 106-5 can be any appropriate shape and size to hold down the sensing element 106-1. To seal the sensing element 106-1 between the bottom layer 106-7 and the top layer 106-4 (i.e., the layer that faces the camera of the mobile device), a sealing device 106-6 is used. For example and without limitation, in some embodiments, the sealing device 106-6 can be an O-ring, sealant, or any other suitable sealing device used to create a seal between the transparent layer 106-5 and the bottom layer 106-7 such that there is a leak-free space for gas exposure to the individual sensors 106-1A through 106-1K.

Referring to FIG. 4C, which illustrates how the gaseous emissions flow through the sensing element 106-1. As shown, once the leak-free space is created the gaseous emissions can ingress into the sensing element 106-1 at the inlet 106-2, react with the individual sensors 106-1A through 106-1K (not shown in this view), and egress the sensing element 106-1 through the outlet 106-3.

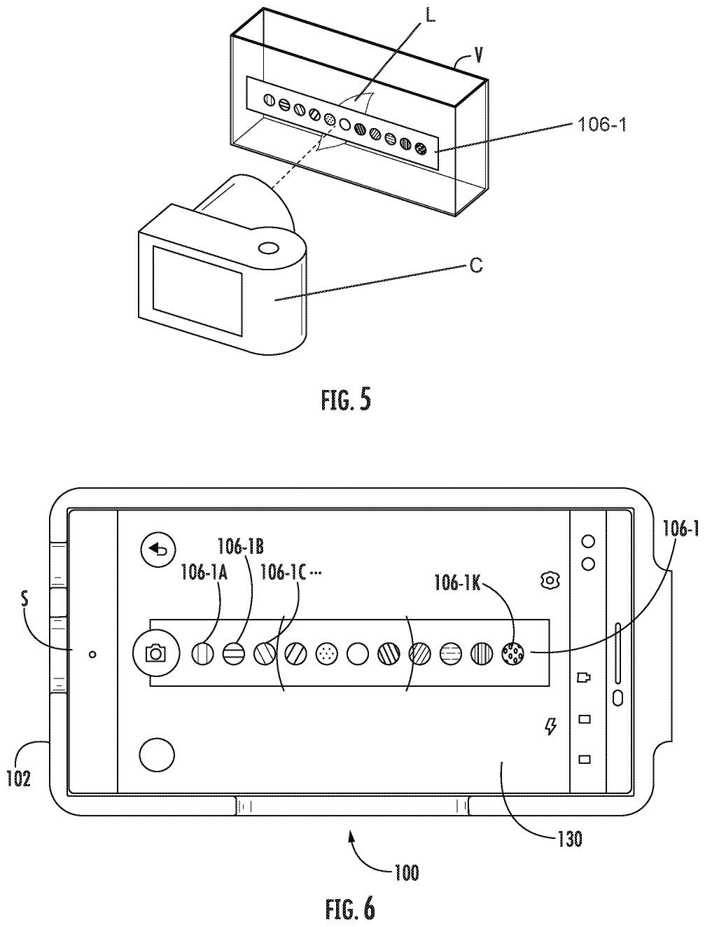

Referring to FIG. 5, which illustrates an alternative embodiment for capturing images of the sensing element 106-1 other than using the receiver cartridge. For example, as illustrated in FIG. 5, the sensing element 106-1 can be placed directly in the vial V or other suitable container (i.e., in a plastic bag, plastic container, glass container, etc.). Once the gases released by the leaf L react with the sensing element 106-1, a mobile device or any other camera C can be used to capture one or more image of the sensing element 106-1 and then the one or more image can be analyzed by one or more processors either integrated with the camera/mobile device C or a separate processor that the camera/mobile device C is capable of transferring the image data to. The one or more processors can analyze the image data according to the image analysis procedures described herein.

Referring to FIG. 6, which illustrates an example user interface 130 of a mobile device S according to some embodiments of the system 100 of the present disclosure. As illustrated in this figure, as the individual sensors 106-1A through 106-1K react with the gaseous emissions, the system is configured such that one or more images can be taken of the sensing element 106-1. Although FIG. 6 illustrates individual sensors 106-1A through 106-1K with hatchings, in a real scenario, the hatchings can be replaced with different colors or intensities (based on the dyes and/or nanoparticles of the individual sensors 106-1A through 106-1K). As the gaseous emissions interact with the individual sensors 106-1A through 106-1K, their color and/or intensity will change if they come into contact with a substance in the gas they are meant to react to. As illustrated on the user interface 130, an operator of the system 100 can press the camera button, capturing one or more images of the sensing element 106-1.

In the systems and methods of the presently disclosed subject matter, the detecting of a signal (e.g., color or fluorescence change, electrical signal, or any other suitable signal, represented by hatching in FIG. 6) from the one or more sensing elements 106-1 can be accomplished by any suitable approach or detector device (i.e., mobile device with a camera or other suitable device) as would be apparent to one of ordinary skill in the art upon a review of the instant disclosure. In some embodiments, detecting a signal comprises capturing one or more images of the one or more sensing elements 106-1 with a camera, such as with a mobile device (i.e., detector) comprising a camera configured to capture one or more image of the receiver cartridge 106. In some embodiments, detecting a signal comprises using a consumer electronics device having a camera configured to capture one or more images of the one or more sensing elements 106-1 and a light source configured to illuminate the one or more sensing elements 106-1 (i.e., the light 124 shown in FIG. 3).

In some embodiments, the systems and methods of the presently disclosed subject matter comprise using a processor for determining the presence and/or the amount of the one or more VOC in the plant sample. In determining the presence and/or the amount of the one or more VOC in the plant sample, the system can then determine the presence of disease, contamination, pest infestation and/or other condition in the plant. By way of example and not limitation, an image captured by a camera can be analyzed with a computer or a smartphone to determine the presence and/or the amount the one or more VOC in the plant sample. For example and without limitation, the processor can analyze the captured image of the dyes or sensors and determine if a pathogen or pest is present by comparing the image to an expected image or expected set of images from a known infection. The processor can further be used to establish a profile of signals for a particular plant condition, e.g., pathogen infection, foodborne contamination, pest infestation, or other condition. For example and without limitation, the system 100 can comprise one or more processors, such as the processor of the mobile device S, configured to determine the presence and/or the amount of the one or more VOC in the plant sample. Additionally, the processor can be an external processor, separate from the mobile device, wherein the mobile device is configured to send the image(s) to the processor for analysis. The established profile can be, for example and without limitation, a series of colors or detected signals that the individual sensors 106-1A through 106-1K give off in the presence of a gaseous emission associated with a pathogen or pest or other tested-for element. In some embodiments, the profiles being generated can comprise profiles that identify and/or distinguish individual species of organism. For example, an infection caused by a particular microbial species can have its own sensor profile, meaning a specific series of colors and shades of the individual sensors 106-1A through 106-1K for that microbial species. As described herein and for example without limitation, in order to determine the amount of VOC present in the plant sample, a parameter called the Euclidean Distance was used. The Euclidean Distance (ED) considers both color and intensity shifts before and after exposure and ED=sqrt (ΔR²+ΔG²+ΔB²), where “sqrt” determines the square root.

In some embodiments, the plant sample is a field sample (e.g. a leaf sample or other sample from another part of the plant) or a sample from a plant product. Thus, the presently disclosed subject matter can also be used to assess plant products, such as seeds, fruits and vegetables, or cut horticultural material for a condition that might develop during transport, such as bacterial or fungal contamination.

In some embodiments, the systems and methods of the presently disclosed subject matter provide for the determining of a condition of the plant or plant product based on the presence and/or the amount of the one or more VOC. In some embodiments, the condition of the plant is an infection by a plant pathogen, including an asymptomatic infection (for example, not apparent upon visual inspection); a contamination by a foodborne microorganism; an abiotic stress condition (for example, a drought condition); a pest (for example, insect) infestation; or any combination thereof. In some embodiments, the infection is an infection caused by a fungus, bacterium, virus, oomycete, or other plant pathogen or pest (e.g., insect pest).

In some embodiments, the systems and methods of the presently disclosed subject matter provide for the generating of a profile of one or more signals from the one or more sensing elements based on the condition of the plant. That is, the profile the one or more signals can be associated with the condition. In some embodiments, generating a profile comprises generating a profile to identify and/or distinguish individual species of organism. In some embodiments, the profile can be used to determine a location of infection or infestation in the plant, such leaves versus roots; to determine a course of treatment for the plant or plant product; and combinations thereof. Early detection facilitates treatment options.

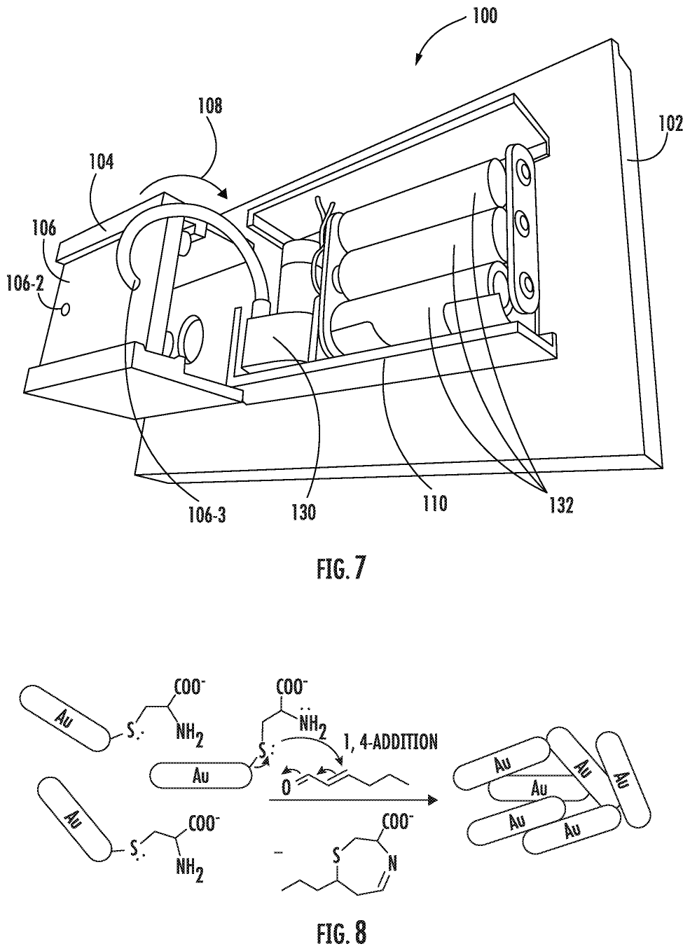

Referring to FIG. 7, which illustrates components of the system 100 of the present disclosure on the back of the mobile device (not visible in this view) and attached to the mobile device case 102. Once the gaseous emission exits the receiver 106 through the outlet 106-3, it enters the micropump chamber 110 due to the sucking force of the micropump 130. Although in the present illustration, the micropump chamber 110 and the micropump 130 are powered by disposable batteries 132, a person having ordinary skill in the art will appreciate that the micropump 130 and the micropump chamber 110 can be powered by any suitable power source. Such power source could be, for example and without limitation, the power source powering the mobile device, an electrical outlet plug into a wall outlet, or any other suitable power source.

In accordance with this disclosure, prototypes and experiments were developed to test various designs and aspects of the system 100. Referring to FIG. 8, which illustrates a mechanism of the aggregation of gold nanorods occurring at the gas-solid interface induced by exposure to (E)-2-Hexenal. An example sensor array was developed, the example sensor array comprising cysteine (Cys)-functionalized gold nanoparticles (Au NPs) or nanorods (Au NRs) as novel plasmonic aggregative colorants for specific recognition of gaseous (E)-2-hexenal, one of the main VOC markers emitted during P. infestans infection of tomato. Upon the exposure to (E)-2-hexenal, Cys is cleaved off from the surface of Au NPs or Au NRs, which induces the aggregation of nanoparticles. The change of inter-nanoparticle distance causes a change of color of the nanosensors. The mechanism of the aggregation of Au NRs occurs at the gas-solid interface induced by exposure to (E)-2-hexenal.

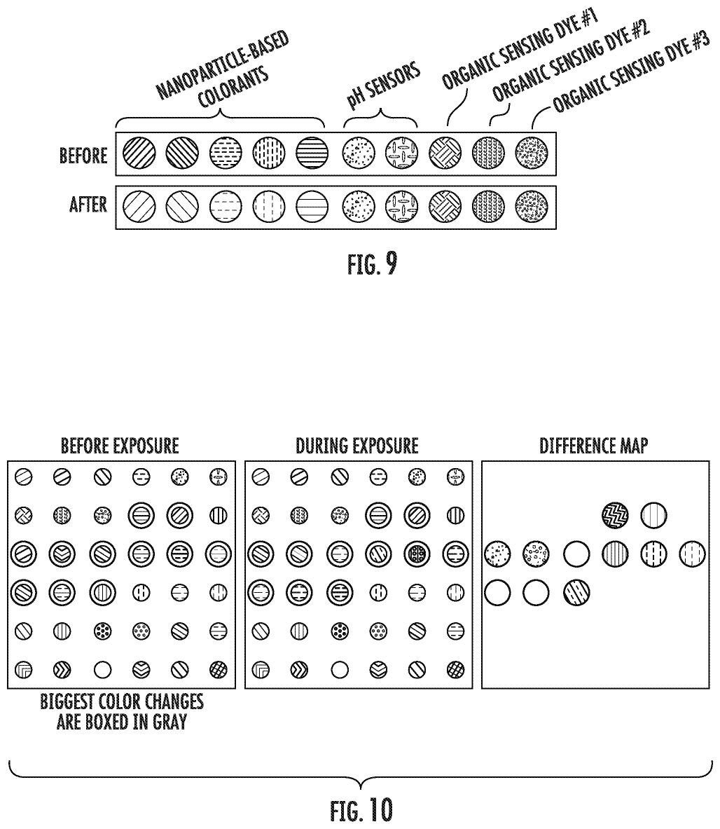

Referring to FIG. 9, which illustrates before- and after-exposure images of a 10-element sensor array in response to 10 ppm (E)-2-hexenal gas. A multiplexed sensor array was developed, combining Cys-functionalized Au nanomaterials and conventional organic colorants for the detection and differentiation of a variety of leaf volatiles. This 10-element colorimetric sensor array contained five representative Au nanomaterials (namely 535-nm and 530-nm Au NPs, 535-nm, 830-nm, and 930-nm Au NRs), along with the other five conventional organic dyes including two pH indicators, two solvatochromic probes, and a generic aldehyde/ketone-sensitive dye. A typical colorimetric sensor array requires the use of multiple cross-reactive dyes to probe a wide range of chemical properties of a single analyte or an analyte “bouquet”. For this particular application, the chemical interactions employed in the example sensor array include Lewis and Brønsted acidity/basicity, molecular polarity, redox property, and solvatochromism associated with plant vapor emissions. Previous research has proved the long shelf-life and good resistance to environmental changes of a similar colorimetric sensor array. In this study, very little variation in sensor response in detection of positive samples (10 ppm (E)-2-hexenal) was observed against a variety of factors, including, humidity, gas flow velocity, temperature, and common interfering agents such as CO₂ and H₂S, which demonstrates the robustness of our sensor array to environmental variation.

The sensor array was then tested with 10 individual plant volatiles, including three GLVs ((Z)-3-hexenal, 1-hexenal, and (E)-2-hexenol), two phytohormones (methyl jasmonate and methyl salicylate), two characteristic late blight markers ((E)-2-hexenal and 2-phenylethanol), and three aromatic VOCs (benzaldehyde, 4-ethylguaiacol, and 4-ethylphenol), to demonstrate the capability for multiplexing. The sensor array was exposed to 10 ppm of each plant volatile and repeated in triplicate. FIG. 9 depicts representative smartphone images of the sensor array before and after exposure to (E)-2-hexenal for 1 min. All Cys-functionalized Au nanomaterials showed distinct and visible color changes after (E)-2-hexenal exposure. Although FIG. 9 shows hatching to depict the colors of the gold nanoparticles and nanorods, pH sensors and other sensors, the key changes between before and after occurred in the gold nanoparticles and nanorod sensors. As can be seen between the depictions of the hatching of the gold nanoparticles and nanorod sensors, the sensors appear lighter in shade after they were exposed to (E)-2-hexenal for 1 min. A change in the intensity of color of the sensors occurs. Although this FIG. 9 depicts a change in hatching, this is meant to portray a change in color shade. For example, between the Before and After row, the nanoparticle-based colorants appear darker in the Before row and lighter in the After row. This is meant to portray a change in color shading from darker shades (Before) of the colorant to lighter shades (After).

Referring to FIG. 10, which visualizes how one or more processors might determine the presence of any particular contaminants, pathogens, or pests in the plant sample. For example, the matrix of shaded dots (although depicted as shaded in FIG. 10, in practice, they would be colored dots) on the far left indicate what the example sensors would look like if they were not exposed to any pathogen, contaminant, or pest. However, if a viewer compares the circled shaded dots in the matrix on the far left to the circled shaded dots in the matrix in the middle, the viewer could readily determine from the picture that all of the circled dots have been altered according to reactions with the gas during exposure. A processor could perform the same function. The processor, given the far left matrix, as a reference, and the middle matrix as the image to compare to the reference, could determine what the differences between the colors are based on the captured picture compared to the reference. The processor could then, for example and without limitation, determine the exact change in the colors, or create a difference map that illustrates the color differences between the reference and the exposed matrix. From the one or more images captured by the mobile device, a matrix of RGB values can be calculated by the processor for each circle and the differences (ΔR, ΔG, ΔB) between the values for each matrix can be calculated to arrive at the difference map on the right. In addition, the Euclidean Distance, defined as ED=sqrt (ΔR²+ΔG²+ΔB²), will be calculated for each circle for quantitative analysis. The ED values of each circle will form a unique sensor response pattern for each VOC or VOC combination. For classification, the ED response pattern will be recognized and differentiated by PCA. Although this is one possible method for determining the differences in the sensors (i.e., depicted by the dots), those having ordinary skill in the art will appreciate that any possible color comparison algorithm or other method such as machine learning is capable of being utilized as well.

Referring to FIG. 11, which illustrates RGB differential profiles of 10 representative plant volatiles at 10 ppm. Although the representative dots (i.e., representing the colors of the sensors) are shaded black and white here, those having ordinary skill in the art will appreciate that the representative dots would be colored in practice, to reflect the dyes and other components of the sensors.

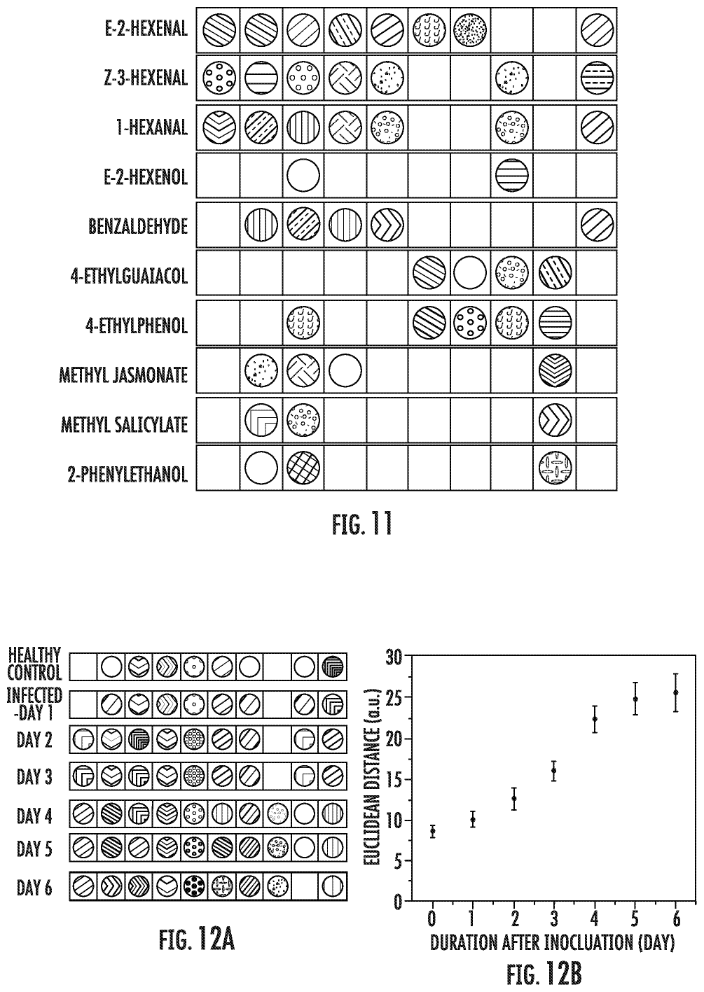

FIG. 12A illustrates RGB differential profiles of volatiles released from infected tomato leaves up to 6 days after inoculation with P. infestans. Although the representative dots (i.e., representing the colors of the sensors) are shaded black and white here, those having ordinary skill in the art will appreciate that the representative dots would be colored in practice, to reflect the dyes and other components of the sensors. FIG. 12B illustrates a response plot showing the Euclidean distance (ED) of all 10 sensor elements as a function of the duration of pathogen infection. The standard deviation represents three independent measurements for each infection duration. A detection threshold for positive samples was set by using the mean ED of healthy control plus three times of its standard deviation. Based on that, the results suggested that the smartphone-based sensing system 100 was able to detect P. infestans as early as the 2^{nd} day after inoculation when symptoms on the plant were not clearly developed yet.

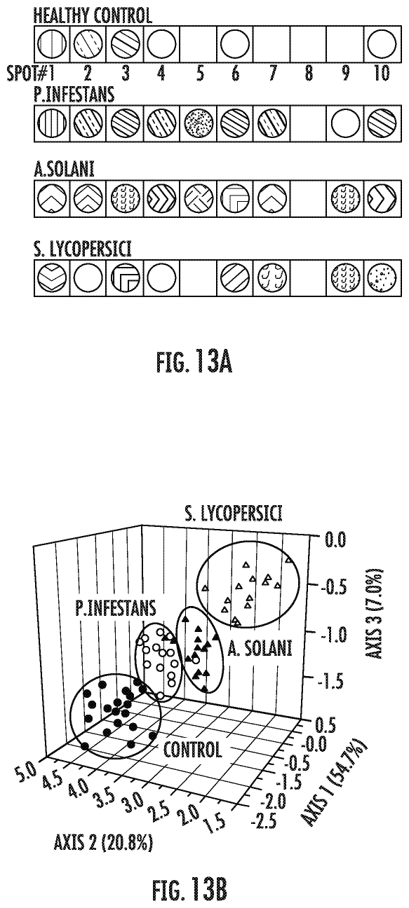

FIG. 13A illustrates differential RGB profiles of uninfected tomato leaves, infected leaves with three pathogens (3 days after inoculation). Although the representative dots (i.e., representing the colors of the sensors) are shaded black and white here, those having ordinary skill in the art will appreciate that the representative dots would be colored in practice, to reflect the dyes and other components of the sensors. FIG. 13B illustrates a PCA plot of infected tomato leaves vs. the healthy control. Each infected species was measured in 15 trials; n=15 biologically independent samples for three infected leaves and n=20 biologically independent samples for the healthy control. These results illustrated the possibility of using the smartphone system to differentiate different plant pathogens with similar symptoms on tomato by VOC profiling.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Introduction to Examples

Plant pathogen detection conventionally relies on molecular technology that is complicated, time consuming, and constrained to centralized laboratories. In accordance with the presently disclosed subject matter, the following Examples relate to the development of a cost-effective smartphone-based volatile organic compound (VOC) fingerprinting platform that allows noninvasive diagnosis of late blight caused by P. infestans by monitoring characteristic leaf volatile emissions in the field. This handheld device integrates a disposable colorimetric sensor array comprising plasmonic nanocolorants and chemo-responsive organic dyes to detect key plant volatiles at the ppm level within one minute of reaction. We demonstrate the multiplexed detection and classification of 10 individual plant volatiles with this field-portable VOC sensing platform, which allows for early detection of tomato late blight 2 days after inoculation, and differentiation from other fungal pathogens of tomato that lead to similar symptoms on tomato foliage. Furthermore, we demonstrate a detection accuracy of >=95% in diagnosis of P. infestans in both lab-inoculated and field-collected tomato leaves in blind pilot tests. Finally, the sensor platform has been beta tested for detection of P. infestans in symptomless tomato plants in the greenhouse setting.

In accordance with the presently disclosed subject matter, the following Examples report a smartphone-integrated plant VOC profiling platform using a paper-based colorimetric sensor array that incorporates functionalized gold nanomaterials and chemo-responsive organic dyes for accurate and early detection of late blight in tomato leaves. In our sensor array, cysteine (Cys)-functionalized gold nanoparticles (Au NPs) or nanorods (Au NRs) were employed as novel plasmonic aggregative colorants for specific recognition of gaseous (E)-2-hexenal, one of the main VOC markers emitted during P. infestans infection¹⁹. Using this handheld device, we demonstrated the identification of 10 common plant volatiles including green leaf volatiles (GLVs) and phytohormones (e.g., methyl jasmonate and methyl salicylate) within one minute of reaction. The multiplexed sensor array was scanned in real time by a 3D-printed smartphone reader and calibrated with known concentrations of plant volatiles to provide quantitative information on volatile mixtures released by healthy and diseased plants. Using an unsupervised pattern recognition method, this smartphone-based VOC sensing platform allows for the sub-ppm detection of (E)-2-hexenal and low-ppm discrimination of a range of disease-related plant VOCs. Finally, the performance of the smartphone device was blind tested using both lab-inoculated tomato leaves and field-collected infected leaves for detection of P. infestans and validated against PCR results.

### Example 1

Development of a Mobile Phone-Based VOC Sensing Platform

We developed a handheld optical scanning platform that integrates a disposable VOC sensor array with the smartphone camera module for digital quantification of relevant plant volatiles. The disposable VOC sensor strips were prepared by deposition of an array of chemical sensors onto nitrocellulose paper substrates. The paper device was placed in the center of the 3D-printed cartridge, and sealed with a microscope cover glass and a rubber O-ring by compression of a sealing cover onto the cartridge to create a leak-free space for gas exposure. The COMSOL simulation of the gas flow in the sensor cartridge showed the superiority of the streamlined gas channel design over other geometries, such as a square-shaped flow chamber design that produced much less uniformity of the flow rate along the gas flow path. The sensor cartridge was inserted into the smartphone attachment and imaged by the camera of the smartphone (FIG. 3).

### Example 2

Nanoplasmonic Materials as Plant Volatile Sensors

The ligand-functionalized plasmonic nanoparticles (NPs) could be used as alternative colorants to organic dyes to detect gaseous analytes of interest. Metallic nanomaterials have been widely used in biological sensing and imaging^{20, 21, 22}. One common sensing mechanism is dependent on changes in localized surface plasmon resonance (LSPR) through the introduction of nanoparticle agglomeration by the binding of target molecules to bio-specific receptors on the nanomaterials. While various aggregation-based colorimetric assays have been developed in solution, very few attempts have been made to trace small gaseous molecules associated with plant pathogens using plasmonic nanomaterials in a dehydrated state. To detect gaseous (E)-2-hexenal, one of the major C₆ GLVs and a reported VOC marker for late blight,¹⁹ we synthesized a series of cysteine (Cys)-capped Au NPs or NRs as LSPR gas sensors. Surface functionalization of nanomaterials was done by the ligand exchange of cetyltrimethylammonium bromide (CTAB) with cysteine. UV-vis spectra of Au NRs exhibited no significant shift in plasmon resonance peaks after cysteine conjugation. FT-IR results clearly indicated the ligation of cysteine to the surface of Au NRs by the detection of characteristic carboxyl (O—C═O) stretching absorption at 1735 cm⁻¹ in Cys-capped Au NR inks. The specific chemical reaction between Cys and (E)-2-hexenal was inspired by the prior work on using α,β-unsaturated carbonyl moiety-conjugated probes for sensitive detection of cysteine or homocysteine^{23, 24}. These functionalized nanomaterials are highly responsive to aliphatic α,β-unsaturated aldehydes via the 1,4-Michael addition reaction, which cleaves the protective Cys ligands off the surface of Au NRs and leads to their aggregation through the formation of a seven-membered ring imine adduct, (3R, S)-7-propyl-2,3,6,7-tetrahydro-1,4-thiazepine-3-carboxylic acid (FIG. 8). The reaction mechanism and byproducts (cleaved molecules) of this reaction were validated by both NMR and MS analyses. UV-vis spectra and TEM results clearly indicate the significant particle aggregation of Au NRs upon exposure to (E)-2-hexenal at 10 ppm level.

We then evaluated the performance of various paper-based Cys-Au NR sensors for effective (E)-2-hexenal detection. Ten Cys-Au NR suspensions with their longitudinal resonant peaks in the range of 530-650 nm were drop-casted on a nitrocellulose paper and dried out as a linear array for gas exposure. The nanoplasmonic sensor array was exposed to different concentrations of (E)-2-hexenal vapors generated from a gas dilution platform. The Cys-Au NR sensors displayed quick reactivity to ppm levels of (E)-2-hexenal vapors, and the reaction equilibrium can be reached within around one minute for analytes at 1 ppm or above. Solid-state Au NRs generally turn purple or gray in response to analytes due to particle aggregation, but the extent of colorimetric responses was highly dependent on the aspect ratio of nanorods. Hypsochromic Au NRs (shorter absorption wavelength range of 530-570 nm) tended to be more responsive with more distinguishable color changes than bathochromic Au NRs (longer absorption wavelength range of 580-650 nm). The results suggest that our low-cost LSPR-based gas sensors can trace hexenal down to the ˜1 ppm level even with the naked eye.

Quantitatively, we determined the limit of detection (LOD) of each Cys-Au NR sensor for detection of (E)-2-hexenal using the Euclidean distance (ED), which is the straight-line distance between two points in the RGB color space (defined as ED=\sqrt{ΔR²+ΔG²+ΔB²}). The LOD was determined by finding the minimum concentration whose corresponding ED value is above the mean of the blank control (i.e., pure N₂ at 50% relative humidity) plus 3λ its standard deviation (3σ). It turns out Cys-Au NR with the UV-vis absorption at 535 nm gives the best LOD of ˜0.4 ppm (Table 3), which is 2 orders of magnitude lower than the vapor concentration of (E)-2-hexenal produced by infected potato tubers as determined by GC-MS (>10 ppm, Table 1)²⁵.

To investigate the effect of particle size and shape in the detection of gaseous aldehyde, eight spherical Au NP suspensions with absorption range of 520-580 nm (particle size of 10-100 nm) and six elongated Au NR inks with longitudinal resonance in the range of 750-930 nm (aspect ratio of 3-6) were prepared, and functionalized with cysteine as previously described. Sensor responses to gaseous C₆ leafy aldehyde were found to be highly dependent on the optical properties of these nanomaterials: for spherical Au NPs, the reactivity gradually decreases with increases of particle size, and the most sensitive response was achieved by 530-nm Au NPs. On the contrary, the response of near infrared (NIR) Au NRs is slightly enhanced with the increase of aspect ratio. Overall, the LODs of spherical Au NPs and NIR Au NRs were not as good as those of short-wavelength Au NRs (Table 3).

### Example 3

Multiplexed Sensor Array for Pattern Identification of Plant Volatiles

We then developed a multiplexed sensor array combining Cys-functionalized Au nanomaterials and conventional organic colorants for the detection and differentiation of a variety of leaf volatiles. This 10-element colorimetric sensor array contains five representative Au nanomaterials (namely 535-nm and 530-nm Au NPs, 535-nm, 830-nm, and 930-nm Au NRs), along with the other five conventional organic dyes including two pH indicators, two solvatochromic probes, and a generic aldehyde/ketone-sensitive dye (FIG. 9 and Table 4). A typical colorimetric sensor array requires the use of multiple cross-reactive dyes to probe a wide range of chemical properties of a single analyte or an analyte “bouquet”^{26, 27}; For this particular application, the chemical interactions employed in our sensor array include Lewis and Brønsted acidity/basicity, molecular polarity, redox property, and solvatochromism associated with plant vapor emissions. Previous research has proved the long shelf-life and good resistance to environmental changes of a similar colorimetric sensor array.²⁸ In this study, we also observed very little variation in sensor response in detection of positive samples (10 ppm (E)-2-hexenal) against a variety of factors, including humidity, gas flow velocity, temperature, and common interfering agents such as CO₂ and H₂S, which demonstrates the robustness of our sensor array to environmental variation.

The sensor array was then tested with 10 individual plant volatiles, including three GLVs ((Z)-3-hexenal, 1-hexenal, and (E)-2-hexenol), two phytohormones (methyl jasmonate and methyl salicylate), two characteristic late blight markers ((E)-2-hexenal and 2-phenylethanol), and three aromatic VOCs (benzaldehyde, 4-ethylguaiacol, and 4-ethylphenol), to demonstrate the capability for multiplexing. The sensor array was exposed to 10 ppm of each plant volatile and repeated in triplicate. FIG. 9 depicts representative smartphone images of the sensor array before and after exposure to (E)-2-hexenal for 1 min. All Cys-functionalized Au nanomaterials showed distinct and visible color changes after (E)-2-hexenal exposure. Readily distinguishable patterns were observed for all 10 plant volatiles tested. We collected response profiles of six representative analytes and calculated their detection limits, which are all well below the diagnostically significant vapor levels as determined by GC-MS on infected plant tissues (Table 1).

Although the LOD is a widely used figure to describe the detection sensitivity of a sensor device, it does not indicate the ability of a sensor to identity a specific analyte in a mixture. The point at which one can discriminate a particular analyte from others is defined as the limit of recognition (LOR), which varies depending on the library of analytes among which a specific target can be differentiated. To determine the LOR of our sensor array, we examined all 10 plant volatiles at 10, 5 and 2.5 ppm. A multivariate technique, principal component analysis (PCA)^{29, 30}, was performed to give a measurement of the dimensionality of the data library. PCA results showed that for the dataset collected at each concentration, it generally requires 5-6 dimensions to account for >95% of total variance for accurate classification. For the simplicity of plotting and visualization, we only use the first three principal components that account for >80% of total variance to display the overall classification. Nine out of ten plant volatiles are perfectly clustered and well separated from the control (N₂ gas) at 10 ppm concentration; In contrast, the volatiles are moderately discriminable at 5 ppm, but indistinguishable at 2.5 ppm. We therefore estimate that the LOR of the sensor array for differentiating main plant volatiles is between 5 and 2.5 ppm, ˜5-10 times higher than their LODs.

### Example 4

Noninvasive Detection of P. infestans

In this Example, the smartphone reader device was modified by incorporating a diaphragm micropump for active sampling of unknown gaseous analytes in the field (FIG. 1). To assess its efficacy for detection of P. infestans-infected plants, fresh tomato leaves were inoculated by spraying 1 mL of P. infestans sporangia suspensions (1,000-10,000 sporangia mL⁻¹) onto the leaf, and their VOC profiles were monitored by the smartphone sensor device daily for up to 6 days after inoculation. Conditions used for pathogen detection were carefully optimized, including accumulation time for headspace gases (60 min) and gas sampling time (1 min). The batch-to-batch reproducibility of disposable volatile test strips was also tested and consistent readout was confirmed. The smartphone-based sensor response patterns of P. infestans-infected tomato where control samples (healthy leaves) showed a relatively weak VOC background. Unique patterns related to potential pathogen infection emerged 2 days after inoculation, and the patterns became more visually distinguishable on subsequent days. Due to the highly mixed nature of the plant leafy volatile emissions, more sensor elements were turned on by the leaf headspace gas compared to previous single VOC species test.

VOC profiles sampled over different times after infection show a steady increase of ED values as a function of days post-inoculation. By applying PCA, infected tomato leaves at varying stages of infection and healthy leaf controls can be readily discriminated by using the first three principal components. Leaf samples profiled 2-4 days after inoculation are clearly clustered and separated from those profiled one day after inoculation or healthy leaf controls, but become indistinguishable at the later stages of infection (5 or 6 days after inoculation) because of the saturation of the sensor signals. Therefore, we conclude that our smartphone-based VOC sensor device is viable for early detection and responds to the infection of P. infestans within 2 days after inoculation prior to visible symptom development.

To demonstrate the specificity for P. infestans detection, we compared the VOC pattern of P. infestans to those of two other fungal pathogens of tomato (Alternaria solani for early blight and Septoria lycopersici for Septoria leaf spot). The volatile composition of leaves inoculated with the three pathogens and the healthy control was first characterized by gas chromatography—mass spectrometry (GC-MS) analysis, which revealed distinguishable VOC signatures of the pathogens from each other. The sensor response profiles of the three pathogens (3 day inoculation) plus a healthy control are shown in FIG. 13A, which displays quantifiable differences in the overall sensor responses. A higher level of (E)-2-hexenal was observed in the cases of P. infestans and A. solani infection as indicated by sensor spots 1-5 and 10, whereas S. lycopersici tended to emit a larger content of 4-ethylphenol and 4-ethylguaiacol that result in higher responses of spot 6 and 7 (FIG. 13A). The sensor responses are generally in a good agreement with the GC-MS measurements. Moreover, a healthy leaf sample spiked with 5 ppm of (E)-2-hexenal produced a VOC response pattern similar to that of a pathogen-infected sample, while other aldehydes (e.g. 1-hexenal) did not respond (FIG. 13A). These results further confirm that (E)-2-hexenal is a major diagnostic VOC marker for P. infestans. Using PCA, we were able to differentiate each of three typical tomato pathogens plus a healthy control with an overall classification accuracy of 95.4% (i.e., only 3 errors out of 65 measurements in total) (FIG. 13B).

Finally, the performance of the smartphone-based VOC sensor was evaluated by two blind tests for detection of P. infestans in both laboratory-inoculated and field-collected leaves, as well as a greenhouse pilot test for continuously monitoring of VOCs from the same tomato plant before and after inoculation over a period of one month. For the double-blinded lab test, 40 anonymous tomato leaf samples were measured on the smartphone VOC sensing platform by personnel who were not involved in sample preparation and PCR validation. The sample pool contained both infected and healthy leaves to challenge the device. PCR tests were run for each sample and used as a standard for validation (Table 5). From the previous tests, we observed that the VOC level of healthy tomato leaves averaged around 10.4±1.2. Therefore, a diagnostic threshold of 14.0, which is the mean of controls plus 3× standard deviation, was chosen for the determination of diseased leaf samples. Using this threshold value, our smartphone VOC sensor was able to rapidly generate binary diagnostic results—positive (+) or negative (−)—on the 40 blind samples tested (Table 5). Only two samples were misdiagnosed by the smartphone VOC sensor, with a detection sensitivity (true positive rate) of 100%, specificity (true negative rate) of 90%, and overall detection accuracy of 95%, when compared to the PCR results (Table 2 and Table 5).

For the blind field sample test, in total 40 tomato leaves were collected, including 20 PCR-positive (+) leaves with suspicious symptoms and 20 symptomless samples (PCR-negative (−)). All infected leaves were collected from tomatoes grown at the Mountain Research Station in Haywood County, N.C. on Aug. 20, 2018³¹. In this pilot study, VOC emissions from all 40 pieces of leaves collected from the field were analyzed using the smartphone VOC detector. Results were then compared side-by-side to results of quantitative PCR (qPCR) following conventional CTAB-based DNA extraction. Out of 20 samples which were identified as positive (+) by qPCR analysis (Table 6), 19 samples were correctly diagnosed by our smartphone VOC sensor, while all negative sample were correctly diagnosed, representing an overall detection sensitivity, specificity, and accuracy of 95%, 100%, and 97.5%, respectively (Table 2). Combining all data together, healthy and infected tomato leaf samples (either lab-inoculated or field-collected) exhibited a clear classification in the PCA plot, based on the first two principal components. The subtle difference between lab-prepared and field-collected infected samples was also captured by the smartphone VOC sensor: lab-inoculated samples displayed a narrower distribution of leafy VOC levels due to better control of the inoculum dose and time, whereas field samples exhibited a wider spread of ED values as a result of the heterogeneous nature of field samples.

For the greenhouse measurements, VOC profiles of healthy leaf controls (three individual tomato plants) were collected once every other day by the smartphone sensor device for 24 days. The plants were then inoculated with P. infestans on the 25th day, and after that the VOCs of infected leaves were monitored daily for another 8 days until the plants completely died. The response curve obtained from this one-month monitoring experiment showed a stable baseline VOC response from healthy tomato plants in the first 24 days, and a rapid increase of VOC emissions 1-2 days after inoculation. These results confirm the ability of the smartphone volatile sensor to capture pathogen-induced leaf volatile changes immediately as infection occurred.

Last but not least, the VOC levels obtained on the smartphone gas sensor from infected samples demonstrated an inversely proportional linear correlation (R²=0.81) to the cycle numbers (Cq) of the P. infestans-specific qPCR assay (Table 6), indicating that higher VOC emission level was associated with higher pathogen DNA content in tomato leaf samples and therefore lower Cq values.

### Discussion of Examples

VOC emission by plants has recently emerged as novel noninvasive diagnostic marker of infectious plant diseases^{32, 33, 34} due to their rich chemical information^{35, 36, 37} and unique functionality in plant self-defense and interplant communications^{38, 39, 40, 41, 42}. Although several portable detection platforms such as electronic noses (e-nose)^{43, 44, 45} have been previously demonstrated for plant volatile analysis, most e-nose technologies only utilize weak chemical interactions, and therefore suffer from several limitations, including: 1) low sensitivity for sub-ppm detection of compounds; 2) limited chemical specificity to discriminate volatiles with similar chemical structures; and 3) severe interference from environmental variation including humidity and temperature.

Alternatively, the presently disclosed smartphone-based VOC sensing method utilizes chemically specific sensing elements comprising cross-reactive plasmonic nanomaterials and dyes with significantly stronger chemical interactions, and therefore results in unprecedented detection sensitivity (Table 1), multiplexity, and chemical selectivity. We also demonstrated that our chemical sensor array is robust and reproducible in signal readout when working under various conditions. Certain toxic gaseous molecules such as H₂S may cause sensor drift (e.g., ˜5% increase in sensor response at 5 ppm of H₂S), which suggests that the use of VOC strips may be limited in certain special scenarios, such as near rotting vegetables or fruits. However, the environment-induced signal drift of VOC strips (<˜5%) is in general much smaller than e-nose sensors (up to 30%)⁴⁵. In addition, the cost of the chemical sensor array is estimated to be ˜15 cents per test, and the smartphone attachment is ˜$20 (excluding the phone), which is orders of magnitude less expensive than commercial e-nose sensors.

Aspects of the presently disclosed subject matter include but are not limited to two areas: first, plasmonic nanostructures are employed as a new class of sensing elements to greatly expand the library of targets that can be analyzed on a conventional chemical sensor array^{28, 46, 47, 48}, and second, a portable mobile phone reader has been integrated to facilitate field deployment and implementation. Although the concept of utilizing localized surface plasmon resonance (LSPR) for gas sensing has been explored by several other groups^{49, 50, 51, 52}, most previous studies rely on bulky and expensive spectrometers for monitoring wavelength shifts or absorption changes, limiting their potential for field applications. Instead, the plasmonic materials in this study are used as chromogenic aggregative colorants embedded in a paper matrix, whose signals—color changes—can be easily detected and quantified by low-cost reader devices such as mobile phones. A mobile app can be employed to conduct image analysis also on the same platform. The detection specificity of plasmonic gas sensors is achieved by the capturing ligands immobilized on the surface of nanostructures, therefore allowing versatile ligand design to extend the applications to a broad range of gaseous targets. On the other hand, despite the great progress in mobile phone-based imaging and sensing technology recently^{53, 54, 55, 56, 57, 58, 59}, only a few applications for gas detection have been demonstrated^{60, 61, 62, 63, 64}, and no mobile phone-based systems have been reported yet for specific, rapid, and noninvasive plant pathogen detection in the field.

The gas sample processing steps in some embodiments of the presently disclosed approach are relatively simple. The use of glass vial for collecting leafy headspace gas from detached samples provides a stable and reproducible testing environment. Moreover, although a 1-h gas accumulation step has been implemented in some embodiments of this initial study, in some embodiments a gas collection time as short as 15 min is employed to differentiate uninfected samples from infected leaves 3-4 days after inoculation. Therefore, sample-to-result times of less than 20 min for field testing are provided in some embodiments of the presently disclosed subject matter. Alternative sampling methods are possible to completely remove the leafy headspace collection step and shorten the total assay time. For example, the sensor patches can be attached directly to the plant leaves for in planta monitoring, where the signals can be continuously received by remote monitoring devices. The wearable design may be more advantageous than smartphone-based scanning in terms of long-term monitoring of symptomless plants and deployment of larger numbers of sensors over a large scale to more efficiently detect early infections in fields. Although we observed that undetached leaves produce 10-15% less volatile emissions than those from detached leaves, such difference may be compensated by better sensor and gas sampling design in future. The current smartphone-based VOC pathogen sensors could be integrated into a disease forecasting system for late blight. They could be used by field extension workers or farmers to trigger a spray event, whereas current late blight forecast systems are mostly weather-based⁶⁵.

In conclusion, in some embodiments the presently disclosed subject matter provides a cost-effective, field-deployable, and integrated VOC sensing platform installed on a smartphone for noninvasive profiling of infectious plant diseases such as late blight with a high degree of detection sensitivity and specificity. The multiplexed chemical sensor assay used in this system is built on plasmonic nanomaterials to target green leafy aldehyde, (E)-2-hexenal, a major late blight VOC marker down to sub-ppm level of LOD. The mobile phone reader device itself integrates bright-field imaging modality, a micro pump for active gas sampling, and wireless connectivity to be used in the field or resource-limited settings. We demonstrated the performance of this portable VOC sensing system for simultaneous detection and classification of 10 individual plant volatiles. By combining this with a pattern classification algorithm such as PCA, diagnosis of tomato late blight as early as 2 days after inoculation was achieved on the mobile phone, which is much earlier than the manifestation of visible symptoms. Moreover, this smartphone-based VOC sensing platform can accurately identify late blight from infected tomato leaf samples either inoculated in the laboratory or collected from the field with a detection accuracy of above 95%. The device has been tested in the greenhouse setting for monitoring of infection progression for a period of one month. Considering the flexibility of sensor array design, multiplexity, and cost-effectiveness, this integrated optical gas sensor platform can be applied to detect other common plant pathogens at very early stages, as well as monitor various abiotic stresses of plants in the field.

### Methods Employed in the Examples

### Reagents and Materials

All reagents and materials were analytical-reagent grade and used without further purification. Reagents for Au nanomaterial synthesis including HAuCl₄, CTAB, AgNO₃, cysteine, NaBH₄ and common solvents were purchased from Sigma-Aldrich (St. Louis, Mo., USA); nitrocellulose membrane (0.45 μm, Cat. No. MCE4547100G) was purchased from Sterlitech Corporation (Kent, Wash., USA); Sensor cartridges were made by 3D printing using a thermoplastic, ABSplus-P430 (Eden Prairie, Minn., USA).

### Preparation of the Smartphone VOC Reader Device

The smartphone attachment and sensor cartridge were designed with Autodesk Inventor, and prototyped using a 3D printer (uPrint SE Plus, Stratasys). The sensor array is illuminated by the default LED flash of the phone (LG V10) and the illumination was uniformed by an optical diffuser (6×9.5×2.3 mm, Parts #02054, Edmund Optics) placed in front of the LED flash. An external lens (12 mm in diameter) with focal distance of 48 mm (Parts #65-576, Edmund Optics) was placed in between the smartphone camera and sensor array to collect the colorimetric signals of the array. The lens provided a demagnification factor of ˜6× (30-mm object distance) so that the entire sensor array could be captured in the field of view of the smartphone reader. The current attachment is designed for an Android smartphone (LG V10), and likewise a similar platform can be easily manufactured for other brands of smartphones such as an iPhone or tablet, after minor modifications to the footprint of the base attachment.

A diaphragm micro pump (T5-1IC-03-1EEP, Parker Hannifin Corp., USA) was installed at the back of the reader device for pulling VOC analytes from real plant tissues onto the sensor array. The micro pump was powered by 3 AA batteries and connected to the sensor cartridge via microtubings (Parts #21564304, Versilon). This battery-powered micro pump generates a gas flow rate of 480 standard cubic centimeter per minute (sccm) to the sensor array.

### Synthesis of Plasmonic Nanomaterials

Short Au NRs: The highly concentrated Au NRs were prepared according to the scale-up, two-step seed-growth method⁶⁶. First, the seeds were made by adding 0.364 g of CTAB to 10 ml of 0.25 mM HAuCl₄. A 0.6 ml of 0.01 M NaBH₄ solution was added dropwise to the above solution thereafter while it was stirring at 800 rpm. The color of the solution instantly became light brown, and the seeds were aged for 5 min and used for all experiments. Second, a two-step seed-growth synthesis was performed: the first growth solution was prepared by mixing HAuCl₄ (0.5 mL, 5 mM), AgNO₃ (8 μL, 0.1 M), ascorbic acid (53 μl, 0.1 M), CTAB (0.364 g), and Milli-Q water (8.5 mL) at room temperature. 1 mL of the seed solution was added into the growth solution and wait for 5 min before further addition of reagents. During the second growth, 100× the concentration of each precursor was added to the solution obtained from the first step, which contained HAuCl₄ (5 mL, 50 mM), AgNO₃ (80 μL, 1 M), ascorbic acid (530 μl, 1 M), CTAB (0.364 g), and Milli-Q water (4.5 mL). The mixture was allowed to react for 10 min before the centrifugation and the collection of the final product. The particle concentration was estimated to be ˜0.02 mM based on the measured optical density and the previously determined extinction coefficients, which was ˜50× as high as that obtained by the conventional seed-mediated method.

Near infrared (NIR) Au NRs: The synthesis of NIR Au NRs follows the same protocol of short Au NRs except that a co-surfactant, benzyldimethylammonium chloride (BDAC), was used along with CTAB in both the first and second steps of seed-mediated Au NR synthesis^{66, 67}. 6 different concentrations of BDAC (0.025, 0.05, 0.075, 0.1, 0.125 and 0.15 mM) were applied that yielded six different NIR Au NRs with absorption wavelength ranging from 750 to 930 nm.

Spherical Au NPs: Spherical Au NPs with different diameters were synthesized by varying the molar ratio of citrate to Au (III) precursor.⁶⁸ Briefly, HAuCl₄ (10 mL, 0.5 mM) was placed in a 50 mL single-neck round flask. The flask was then immersed in an oil bath without reflux and heated to 100° C. under vigorous stirring at 800 rpm for 10 min. While the Au (III) solution is boiling, different volumes (0.25, 0.5, 0.5, 1.25, 2, 4, 7 and 12 mL) of citrate solutions (5 mM) preheated at the reaction temperature were quickly added in. The product was allowed to cool down to room temperature after the reaction proceeded for another 10 min, centrifuged and washed 3× and then dissolved in 0.2 mL nanopure water to make it ˜50× as concentrated as the initially obtained Au NP solution.

### Oxidation and Ligand Exchange of Nanoplasmonic Materials

For particle oxidation, different amounts (10-100 μL) of a mild oxidant, HAuCl₄ (5 mM), were added to the Au NR solution⁶⁹. The oxidation process occurred 5 min after the addition of Au(III), which was monitored by a UV-vis spectrometer to record the extinction spectra over time. Once each of the 10 desired longitudinal plasmon resonance wavelengths (530-650 nm) were achieved, the oxidation process was stopped by precipitating Au NRs with centrifugation and redispersing them in 0.1 M CTAB solution. The aspect ratio (AR) of Au NRs were tuned in between 1-2.5, which produces nanorods with an average width of 20 nm and varied length from 20 to 50 nm, as evidenced by TEM images. For ligand exchange, 1 mL of 0.1 M cysteine was added to 1 mL CTAB-capped Au NR solution, and the mixture was stirred at room temperature for 24 h. The final products were collected with centrifugation and redispersed in 0.1 M cysteine prior to the preparation of sensor arrays.

### Characterization of Au Nanomaterials

For the studies of surface chemistry and nanoparticle morphologies, FT-IR spectra were acquired on a Perkin Elmer Frontier spectrometer from 4000 cm⁻¹ to 1000 cm⁻¹. UV-vis absorption data was collected on a Thermo Evolution 201 UV-vis spectrophotometer. TEM was performed on a JEOL 2000FX with an acceleration voltage of 200 kV.

For the validation of chemical reaction mechanism during nanoparticle aggregation, 1:1 molar mixture of cysteine (3.02 g, 25 mmol) and (E)-2-hexenal (2.45 g, 25 mmol) were dissolved in D₂O (20 mL) and stirred at room temperature for 2 h to simulate the gas-phase sensing reaction. The solid were filtered, washed with D₂O and dried under vacuum to give the white product (4.22 g, yield 84%). NMR solution was prepared by redissolving the purified product (20 mg) in D₂O (0.75 mL) and DCl (0.05 mL). ¹H and ¹³C NMR spectra of the as-synthesized product were recorded on a Varian 600 MHz spectrometer. ¹H NMR (600 MHz, D₂O): δ 7.5 (dd, 1H), 4.63 (s, 1H, solvent), 4.02 (dd, 1H), 3.47 (dd, 1H), 3.24 (dd, 2H), 2.48 (ddd, 2H), 1.52 (dd, 2H), 1.40 (dd, 2H), 1.06 (t, 3H); ¹³C NMR (125 MHz, D₂O): δ 173.9, 67.4, 65.7, 47.6, 47.1, 35.8, 32.4, 19.6, 17.2. Mass spectra were collected on a Waters Q-TOF Premier Mass Spectrometer. ESI-MS m/z: calculated C₉H₁₅O₂NS [M+H]⁺=202.0; detected=201.9. Both NMR and MS results support the formation of seven-membered ring imine product, (3R, S)-7-propyl-2,3,6,7-tetrahydro-1,4-thiazepine-3-carboxylic acid, as the major product during the nanoparticle aggregation.

### Sensor Array Preparation

Each of the Au nanomaterial inks was used as is, while the other five organic dyes were prepared in the sol-gel formulations (in porous silica made from the hydrolysis of tetraethoxysilane and ethyltriethoxysilane, as reported previously⁴⁶). ˜150 nL of each Au nanomaterial ink or dye formulation was transferred by slotted stainless steel pins (Parts #FP4CB, V&P Scientific) and drop casted onto the nitrocellulose substrate to form a round colored spot with ˜1 mm in diameter, using a LEGATO® 180 picoliter syringe pump (KD Scientific Inc., Holliston, Mass.). Detailed composition and concentration of each sensor element can be found in Table 4. Before the measurements, colorimetric sensor arrays were stored in a nitrogen filled desiccator for 24 h. The sensor arrays are stable for 1 month under storage in N₂.

### Gas Exposure and Image Capturing Experiment

Gas mixtures were prepared according to previous methods⁴⁵. Briefly, MKS mass flow controllers were used to achieve gas streams with the desired concentration (e.g., 0.1-100 ppm of (E)-2-hexenal), flow rate (500 sccm) and relative humidity (50% RH) by mixing the proper portion of saturated vapor of the liquid analyte with dry (0% RH) and wet (100% RH) nitrogen gas. Arrays were exposed to a control stream (50% RH N₂) for 1 min followed by 1 min exposure of an analyte stream. A photo was taken by the camera of a smartphone, LG V10, at the end of 1 min exposure to either the control or the analyte, as the before- or after-exposure image.

### Inoculation of Tomato Leaves and Detection of Headspace Gas

Tomato seedlings were purchased from local supermarket and cultivated in a greenhouse at 25±3° C. under 16 h of light per day. A typical P. infestans strain (NC 14-1, US-23) was cultured on rye medium in the dark at 20° C. Leaves collected from tomato plants at the five to six leaf stage were inoculated with suspensions of P. infestans sporangia (˜10000 sporangia mL⁻¹) in a sterile acid-washed Petri dish (100×15 mm). Healthy tomato leaves treated with sterile water were used as controls and kept under the same condition. The infected leaves and the control leaves were quickly transferred into borosilicate scintillation vial (20 mL) with screw lids and incubated at room temperature with 95% relative humidity. The capped vials were further sealed with Parafilm (Bemis Company, Neenah, Wis.) to allow the headspace gases to accumulate for 1 h prior to the measurement. The headspaces above each of the infected leaf samples and the controls were sampled by the micro pump-equipped smartphone VOC sensing device every 24 h after inoculation over the next several days.

SPME GC/MS test of plant volatiles

Solid-phase microextraction (SPME) sampling was performed using non-polar divinylbenzene/carboxen/polydimethylsiloxane (DVB/CAR/PDMS) fibers. The screw thread SPME vials were fitted with Teflon septa and loaded with a healthy leaf or each of the three inoculated leaves to accumulate the vapor for 1 h. The fiber then penetrated into the septa to extract the volatiles for 2 min. GC/MS experiments were carried out using an Agilent Technologies 7890A GC/MS equipped with a flame ionization detector (FID) and mass selective detector. The injector temperature was kept at 80° C. and analytes were desorbed for 2 min. The carrier gas was helium (1 mL/min). For analysis, the initial oven temperature was maintained at 80° C. for 2 min, increased at a ramp rate of 5° C./min to 305° C. for 45 min. The GC-MS built-in NIST libraries were used to interpret the mass spectra.

qPCR Analysis of Field Leaf Samples

For CTAB-based DNA extraction, approximately 10 mg homogenized leaf sample was taken in a microcentrifuge tube and mixed with 150 μl extraction buffer (0.35 M sorbitol, 0.1 M Tris, 0.005 M EDTA, 0.02 M sodium bisulfite, pH 7.5), 150 μL nuclei lysis buffer (0.2 M Tris, 0.05 M EDTA, 2.0 M NaCl, and 2% CTAB, pH 7.5), and 60 μL 5% N-lauryl sarcosine. Then, the tube was incubated at 65° C. for 30 min. After incubation, 300 μL chloroform was added to the tube and centrifuged at 12000 rpm. The aqueous phase containing DNA was transferred to a new tube and mixed with 300 μL cold isopropanol (100%) and 30 μL 3M sodium acetate (pH 8). The sample was stored overnight at −20° C., and then centrifuged at 13000 rpm for 5 min to pellet the precipitated DNA. After discarding the supernatant, 1 mL cold ethanol (70%) was added to wash the pellet. The sample was centrifuged again at 13000 rpm for 5 min and the ethanol solution was disposed. Finally, the DNA pellet was air dried in a fume hood and resuspended in 100 μL TE buffer (10 mM Tris-HCl, 0.1 mM EDTA, pH 8.0). For qPCR amplification, 1 μL template DNA was used with two P. infestans specific primers PINF (CTCGCTACAATAGGAGGGTC; SEQ ID NO:1) and HERB1 (CGGACCGCCTGCGAGTCC; SEQ ID NO:2), which generate an amplicon length of ˜100 bp using a previously published thermocycling procedure⁷⁰.

### Greenhouse Measurements

Three robust tomato plants grown in the pots were placed in a clear plastic bin and cultivated in the greenhouse under room temperature, with 12 h illumination per day. A damp paper towel at the bottom of the bin was used to keep high relative humidity. The VOC level of each plant during the healthy growth phase was monitored and recorded daily over 24 days. At the 25th day, 1 mL of sporangia solution (5×10³ sporangia/mL) was evenly misted onto the leaves of plants, and the lid of the bin was completely closed to allow for 100% RH and to avoid spreading of the pathogen. Symptoms of late blight became apparent 3 days after inoculation. The VOC level was continuously monitored by the smartphone detector until the 8^{th} day after inoculation, when the complete death of the plants was occurred.

TABLE 1
Limit of detections of six representative plant volatiles
detected by the chemical sensor array on the smartphone,
as compared to the vapor levels detected in P. infestans-infected
potato tissues by GC-MS.

| | LOD of the Smartphone | Vapor level determined |
|---|---|---|
| Plant VOCs | VOC sensor (ppm) | by GC-MS (ppm) ^{a} |
| (E)-2-Hexenal | 0.4 | 12-18 |
| (Z)-3-hexenal | 1.1 | 6-12 |
| 1-Hexanal | 1.7 | 3-6 |
| 4-Ethylphenol | 1.8 | 3-6 |
| Benzaldehyde | 0.9 | 0.3-1.5 |
| 2-Phenylethanol | 5.2 | 1.5-3 |

^{a} Data is recalculated from Ref. ²⁵ De Lacy Costello, B. P. et al. Plant Pathology 2001, 50, 489-496.

TABLE 2
Quantification of the detection sensitivity, specificity,
and accuracy of the smartphone VOC sensor in the blind
tests, based on PCR results used as the gold standard.

| | Blind Lab Samples | | Blind Field Samples | |
|---|---|---|---|---|
| | (n = 40) | | (n = 40) | |
| | PCR | VOC | qPCR | VOC |
| True Positive (TP) | 20 | 20 | 20 | 19 |
| False Positive (FP) | — | 2 | — | 0 |
| True Negative (TN) | 20 | 18 | 20 | 20 |
| False Negative (FN) | — | 0 | — | 1 |
| Sensitivity (TP/P) | — | 100% | — | 95% |
| Specificity (TN/N) | — | 90% | — | 100% |
| Accuracy ((TP + TN)/n)) | — | 95% | — | 97.5% |

TABLE 3
Comparison of limits of detection (LODs) for (E)-2-hexenal
using different Cys-capped Au nanomaterials as sensors.

| Short Au NRs | | NIR Au NRs | | Spherical Au NPs | |
|---|---|---|---|---|---|
| Wavelength | LOD | Wavelength | LOD | Wavelength | LOD |
| (nm) | (ppm) | (nm) | (ppm) | (nm) | (ppm) |
| 650 | 2.4 | 750 | 2.8 | 580 | 1.4 |
| 630 | 1.7 | 770 | 1.9 | 565 | 1.1 |
| 605 | 1.4 | 790 | 1.1 | 550 | 0.9 |
| 590 | 1.4 | 830 | 0.84 | 535 | 0.66 |
| 580 | 1.3 | 870 | 0.96 | 530 | 0.53 |
| 570 | 0.92 | 930 | 1.2 | 525 | 0.78 |
| 560 | 1.1 | | | 522 | 0.84 |
| 550 | 0.65 | | | 520 | 0.97 |
| 535 | 0.42 | | | | |
| 530 | 0.59 | | | | |

TABLE 4
Composition of the 10-element colorimetric sensor array

| Spot # | Composition | Amount |
|---|---|---|
| 1 | AuNP@535 nm | Use as is |
| 2 | AuNP@530 nm | Use as is |
| 3 | AuNR@535 nm | Use as is |
| 4 | AuNR@830 nm | Use as is |
| 5 | AuNR@930 nm | Use as is |
| 6 | Bromothymol Blue + | 2 mg + 50 μL + |
| | Tetrabutylammonium Hydroxide | 1.5 g + 1 mL |
| | (1M) + Silica + sol-gel | |
| 7 | Chlorophenol Red + | 2 mg + 50 μL + |
| | Tetrabutylammonium Hydroxide | 1.5 g + 1 mL |
| | (1M) + Silica + sol-gel | |
| 8 | Reichardt's Dye + Silica + sol-gel | 2 mg + 1.5 g + 1 mL |
| 9 | Merocyanine 540 + Silica + sol-gel | 2 mg + 1.5 g + 1 mL |
| 10 | Pararosaniline Acetate + 2,4- | 1 mg + 10 mg + 25 |
| | Dinitrophenylhydrazine + H₂SO₄ | μL + 1.5 g + 1 mL |
| | (1M) + Silica + sol-gel | |

TABLE 5
Blind pilot test of 20 lab-inoculated and 20 healthy samples arranged
in a random order. All predictions are correct by the smartphone
except sample #4; the overall accuracy is 38/40, or 95%.

| Sample # | ED Value (a.u.) | VOC Prediction | PCR Identification |
|---|---|---|---|
| 1 | 11.3 | − | − |
| 2 | 9.9 | − | − |
| 3 | 10.6 | − | − |
| 4 | 15.4 | + | − |
| 5 | 21.2 | + | + |
| 6 | 18.9 | + | + |
| 7 | 12.0 | − | − |
| 8 | 10.8 | − | − |
| 9 | 17.9 | + | + |
| 10 | 20.1 | + | + |
| 11 | 19.6 | + | + |
| 12 | 11.1 | − | − |
| 13 | 12.3 | − | − |
| 14 | 18.6 | + | + |
| 15 | 11.4 | − | − |
| 16 | 17.5 | + | + |
| 17 | 18.8 | + | + |
| 18 | 17.9 | + | + |
| 19 | 10.7 | − | − |
| 20 | 19.0 | + | + |
| 21 | 20.2 | + | + |
| 22 | 18.3 | + | + |
| 23 | 12.6 | − | − |
| 24 | 17.8 | + | + |
| 25 | 14.7 | + | − |
| 26 | 10.9 | − | − |
| 27 | 11.0 | − | − |
| 28 | 17.5 | + | + |
| 29 | 10.7 | − | − |
| 30 | 19.2 | + | + |
| 31 | 12.1 | − | − |
| 32 | 18.0 | + | + |
| 33 | 11.8 | − | − |
| 34 | 10.5 | − | − |
| 35 | 16.8 | + | + |
| 36 | 19.0 | + | + |
| 37 | 11.3 | − | − |
| 38 | 18.4 | + | + |
| 39 | 18.1 | + | + |
| 40 | 10.2 | − | − |

TABLE 6
VOC and qPCR analysis of blind field samples and greenhouse
infected samples. Field tomato leaves were collected from
the Mountain Research Station and greenhouse samples were
from the Phytotron Laboratory of NC State University. The
cycle number (Cq) is generally inversely related to the overall
VOC level (i.e., magnitude of Euclidean distance).

| Field infected samples | | | Greenhouse infected samples | | |
|---|---|---|---|---|---|
| Sample ID | Cq | ED (a.u.) | Sample ID | Cq | ED (a.u.) |
| 1 | 21.4 | 14.3 | 1 | 24.1 | 13.3 |
| 2 | 21.0 | 16.9 | 2 | 23.8 | 13.6 |
| 3 | 22.0 | 19.2 | 3 | 23.4 | 13.4 |
| 4 | 22.8 | 18.4 | 4 | 22.2 | 14.7 |
| 5 | 22.5 | 22.7 | 5 | 22.0 | 15.4 |
| 6 | 19.8 | 24.3 | 6 | 21.8 | 15.8 |
| 7 | 23.9 | 13.5 | 7 | 20.4 | 18.9 |
| 8 | 21.8 | 21.2 | 8 | 20.1 | 19.7 |
| 9 | 19.5 | 23.5 | 9 | 19.8 | 20.2 |
| 10 | 19.6 | 19.6 | 10 | 19.4 | 23.6 |
| 11 | 20.1 | 20.1 | 11 | 19.2 | 24.1 |
| 12 | 21.0 | 16.8 | 12 | 19.9 | 23.8 |
| 13 | 21.2 | 21.3 | 13 | 18.7 | 25.3 |
| 14 | 19.6 | 18.9 | 14 | 18.5 | 25.9 |
| 15 | 22.3 | 15.6 | 15 | 18.4 | 26.0 |
| 16 | 22.5 | 22.9 | 16 | 17.7 | 26.9 |
| 17 | 19.5 | 25.6 | 17 | 18.0 | 26.5 |
| 18 | 20.4 | 19.0 | 18 | 17.8 | 26.1 |
| 19 | 19.3 | 22.1 | 19 | 18.6 | 25.4 |
| 20 | 22.6 | 17.4 | 20 | 18.3 | 25.9 |

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Oerke E C. Crop losses to pests. The Journal of Agricultural Science 144, 31-43 (2006). or compositions employed herein.
2. Pimentel D, Lach L, Zuniga R, Morrison D. Environmental and Economic Costs of Nonindigenous Species in the United States. BioScience 50, 53-65 (2000).
3. Nowicki M, Foolad M R, Nowakowska M, Kozik E U. Potato and Tomato Late Blight Caused by Phytophthora infestans: An Overview of Pathology and Resistance Breeding. Plant Disease 96, 4-17 (2011).
4. Saville A C, Martin M D, Ristaino J B. Historic Late Blight Outbreaks Caused by a Widespread Dominant Lineage of Phytophthora infestans (Mont.) de Bary. PLOS ONE 11, e0168381 (2016).
5. Pennisi E. Armed and Dangerous. Science 327, 804-805 (2010).
6. Haverkort A J, Struik P C, Visser R G F, Jacobsen E. Applied Biotechnology to Combat Late Blight in Potato Caused by Phytophthora Infestans. Potato Research 52, 249-264 (2009).
7. Fry W E, et al. The 2009 Late Blight Pandemic in the Eastern United States—Causes and Results. Plant Dis 97, 296-306 (2013).
8. Hussain S, Lees A K, Duncan J M, Cooke D E L. Development of a species-specific and sensitive detection assay for Phytophthora infestans and its application for monitoring of inoculum in tubers and soil. Plant Pathology 54, 373-382 (2005).
9. Lees A K, Sullivan L, Lynott J S, Cullen D W. Development of a quantitative real-time PCR assay for Phytophthora infestans and its applicability to leaf, tuber and soil samples. Plant Pathology 61, 867-876 (2012).
10. Khan M, Li B, Jiang Y, Weng Q, Chen Q. Evaluation of Different PCR-Based Assays and LAMP Method for Rapid Detection of Phytophthora infestans by Targeting the Ypt1 Gene. Front Microbiol 8, 1920 (2017).
11. Hansen Z R, et al. Loop-mediated isothermal amplification for detection of the tomato and potato late blight pathogen, Phytophthora infestans. J Appl Microbiol 120, 1010-1020 (2016).
12. Bodrossy L, Sessitsch A. Oligonucleotide microarrays in microbial diagnostics. Curr Opin Microbiol 7, 245-254 (2004).
13. Wakeham A J, Keane G, Kennedy R. Field Evaluation of a Competitive Lateral-Flow Assay for Detection of Alternaria brassicae in Vegetable Brassica Crops. Plant Disease 100, 1831-1839 (2016).
14. Harrison J G, Lowe R, Duncan J M. Use of ELISA for assessing major gene resistance of potato leaves to Phytophthora infestans. Plant Pathol 40, 431-435 (1991).
15. Skottrup P, Nicolaisen M, Justesen A F. Rapid determination of Phytophthora infestans sporangia using a surface plasmon resonance immunosensor. Journal of Microbiological Methods 68, 507-515 (2007).
16. Ray M, et al. Fungal disease detection in plants: Traditional assays, novel diagnostic techniques and biosensors. Biosens Bioelectron 87, 708-723 (2017).
17. Koo C, et al. Development of a Real-Time Microchip PCR System for Portable Plant Disease Diagnosis. PLOS ONE 8, e82704 (2013).
18. Julich S, et al. Development of a lab-on-a-chip device for diagnosis of plant pathogens. Biosens Bioelectron 26, 4070-4075 (2011).
19. Laothawornkitkul J, Jansen R M C, Smid H M, Bouwmeester H J, Muller J, van Bruggen A H C. Volatile organic compounds as a diagnostic marker of late blight infected potato plants: A pilot study. Crop Protection 29, 872-878 (2010).
20. Chen G, Roy I, Yang C, Prasad P N. Nanochemistry and Nanomedicine for Nanoparticle-based Diagnostics and Therapy. Chem Rev 116, 2826-2885 (2016).
21. Sabela M, Balme S, Bechelany M, Janot J-M, Bisetty K. A Review of Gold and Silver Nanoparticle-Based Colorimetric Sensing Assays. Advanced Engineering Materials 19, 1700270 (2017).
22. Yu T, Wei Q. Plasmonic molecular assays: Recent advances and applications for mobile health. Nano Res 11, 5439-5473 (2018).
23. Li H, et al. A fluorescent chemodosimeter specific for cysteine: effective discrimination of cysteine from homocysteine. Chem Commun, 5904-5906 (2009).
24. Wang W, et al. Detection of Homocysteine and Cysteine. J Am Chem Soc 127, 15949-15958 (2005).
25. De Lacy Costello B P J, et al. Gas chromatography-mass spectrometry analyses of volatile organic compounds from potato tubers inoculated with Phytophthora infestans or Fusarium coeruleum. Plant Pathology 50, 489-496 (2001).
26. Li Z, Bassett W P, Askim J R, Suslick K S. Differentiation among peroxide explosives with an optoelectronic nose. Chem Commun 51, 15312-15315 (2015).
27. Li Z, Fang M, LaGasse M K, Askim J R, Suslick K S. Colorimetric Recognition of Aldehydes and Ketones. Angew Chem Int Ed 56, 9860-9863 (2017).
28. Li Z, Suslick K S. Portable Optoelectronic Nose for Monitoring Meat Freshness. ACS Sensors 1, 1330-1335 (2016).
29. Janata J. Principles of Chemical Sensors, 2^{nd} Edition. Springer (2009).
30. Tabora J E, Domagalski N. Multivariate Analysis and Statistics in Pharmaceutical Process Research and Development. Annu Rev Chem Biomol Eng 8, 403-426 (2017).
31. Meadows I. Late Blight Detected in Haywood County—Aug. 20, 2018. https://plantpathology.ces.ncsu.edu/2018/08/late-blight-detected-in-haywood-county-aug-20-2018/.
32. Jansen R M C, Hofstee J W, Wildt J, Verstappen F W A, Bouwmeester H J, van Henten E J. Induced plant volatiles allow sensitive monitoring of plant health status in greenhouses. Plant Signaling & Behavior 4, 824-829 (2009).
33. Jansen R M C, Wildt J, Kappers I F, Bouwmeester H J, Hofstee J W, van Henten E J. Detection of Diseased Plants by Analysis of Volatile Organic Compound Emission. Annual Review of Phytopathology 49, 157-174 (2011).
34. Aksenov A A, et al. Volatile Organic Compounds (VOCs) for Noninvasive Plant Diagnostics. American Chemical Society (2013).
35. Dudareva N, Negre F, Nagegowda D A, Orlova I. Plant Volatiles: Recent Advances and Future Perspectives. Critical Reviews in Plant Sciences 25, 417-440 (2006).
36. Matsui K. Green leaf volatiles: hydroperoxide lyase pathway of oxylipin metabolism. Current Opinion in Plant Biology 9, 274-280 (2006).
37. Holopainen J, Blande J. Where do herbivore-induced plant volatiles go? Frontiers in Plant Science 4, 185 (2013).
38. Paré PW, Tumlinson J H. Plant Volatiles as a Defense against Insect Herbivores. Plant Physiology 121, 325-332 (1999).
39. Marcel D. Behavioural and community ecology of plants that cry for help. Plant, Cell & Environment 32, 654-665 (2009).
40. Holopainen J K, Gershenzon J. Multiple stress factors and the emission of plant VOCs. Trends in Plant Science 15, 176-184 (2010).
41. Scala A, Allmann S, Mirabella R, Haring M, Schuurink R. Green Leaf Volatiles: A Plant's Multifunctional Weapon against Herbivores and Pathogens. International Journal of Molecular Sciences 14, 17781 (2013).
42. Erb M. Volatiles as inducers and suppressors of plant defense and immunity—origins, specificity, perception and signaling. Current Opinion in Plant Biology 44, 117-121 (2018).
43. Laothawornkitkul J, et al. Discrimination of Plant Volatile Signatures by an Electronic Nose: A Potential Technology for Plant Pest and Disease Monitoring. Environmental Science and Technology 42, 8433-8439 (2008).
44. Wilson A. Diverse Applications of Electronic-Nose Technologies in Agriculture and Forestry. Sensors 13, 2295 (2013).
45. Cellini A, Blasioli S, Biondi E, Bertaccini A, Braschi I, Spinelli F. Potential Applications and Limitations of Electronic Nose Devices for Plant Disease Diagnosis. Sensors 17, 2596 (2017).
46. Li Z, Jang M, Askim J R, Suslick K S. Identification of accelerants, fuels and post-combustion residues using a colorimetric sensor array. Analyst 140, 5929-5935 (2015).
47. Li Z, Suslick K S. A Hand-Held Optoelectronic Nose for the Identification of Liquors. ACS Sensors 3, 121-127 (2018).
48. Askim J R, Mahmoudi M, Suslick K S. Optical sensor arrays for chemical sensing: the optoelectronic nose. Chem Soc Rev 42, 8649-8682 (2013).
49. Bingham J M, Anker J N, Kreno L E, Van Duyne R P. Gas Sensing with High-Resolution Localized Surface Plasmon Resonance Spectroscopy. J Am Chem Soc 132, 17358-17359 (2010).
50. Liu N, Tang M L, Hentschel M, Giessen H, Alivisatos A P. Nanoantenna-enhanced gas sensing in a single tailored nanofocus. Nature Materials 10, 631 (2011).
51. Yang Z, Sassa F, Hayashi K. A Robot Equipped with a High-Speed LSPR Gas Sensor Module for Collecting Spatial Odor Information from On-Ground Invisible Odor Sources. ACS Sensors 3, 1174-1181 (2018).
52. Shang L, Liu C, Chen B, Hayashi K. Plant biomarker recognition by molecular imprinting based LSPR sensor array: Performance improvement by enhanced hotspot of Au nanostructure. ACS Sensors 3, 1531-1538 (2018).
53. Ozcan A. Mobile phones democratize and cultivate next-generation imaging, diagnostics and measurement tools. Lab Chip 14, 3187-3194 (2014).
54. Contreras-Naranjo J C, Wei Q, Ozcan A. Mobile Phone-Based Microscopy, Sensing, and Diagnostics. IEEE J Sel Top Quantum Electron 22, 1-14 (2016).
55. Wei Q, et al. Fluorescent Imaging of Single Nanoparticles and Viruses on a Smart Phone. ACS Nano 7, 9147-9155 (2013).
56. Wei Q, et al. Imaging and Sizing of Single DNA Molecules on a Mobile Phone. ACS Nano 8, 12725-12733 (2014).
57. Joh D Y, et al. Inkjet-printed point-of-care immunoassay on a nanoscale polymer brush enables subpicomolar detection of analytes in blood. Proc Natl Acad Sci 114, E7054-E7062 (2017).
58. Kühnemund M, et al. Targeted DNA sequencing and in situ mutation analysis using mobile phone microscopy. Nat Commun 8, 13913 (2017).
59. Hernandez-Neuta I, et al. Smartphone-based clinical diagnostics: towards democratization of evidence-based health care. J Intern Med 285, 19-39 (2019).
60. Ninh H P, Tanaka Y, Nakamoto T, Hamada K. A bad-smell sensing network using gas detector tubes and mobile phone cameras. Sensor Actuator B Chem 125, 138-143 (2007).
61. Azzarelli J M, Mirica K A, Ravnsbæk JB, Swager T M. Wireless gas detection with a smartphone via rf communication. Proc Natl Acad Sci 111, 18162-18166 (2014).
62. Salles M O, Meloni G N, de Araujo W R, Paixão TRLC. Explosive colorimetric discrimination using a smartphone, paper device and chemometrical approach. Anal Methods 6, 2047-2052 (2014).
63. Gahlaut S K, Yadav K, Sharan C, Singh J P. Quick and Selective Dual Mode Detection of H₂S Gas by Mobile App Employing Silver Nanorods Array. Anal Chem 89, 13582-13588 (2017).
64. Devadhasan J P, Kim D, Lee D Y, Kim S. Smartphone coupled handheld array reader for real-time toxic gas detection. Anal Chim Acta 984, 168-176 (2017).
65. Small I M, Joseph L, Fry W E. Evaluation of the BlightPro Decision Support System for Management of Potato Late Blight Using Computer Simulation and Field Validation. Phytopathology 105, 1545-1554 (2015).
66. Park K, et al. Highly Concentrated Seed-Mediated Synthesis of Monodispersed Gold Nanorods. ACS Appl Mater Interfaces 9, 26363-26371 (2017).
67. Nikoobakht B, El-Sayed M A. Preparation and Growth Mechanism of Gold Nanorods (NRs) Using Seed-Mediated Growth Method. Chem Mater 15, 1957-1962 (2003).
68. FRENS G. Controlled Nucleation for the Regulation of the Particle Size in Monodisperse Gold Suspensions. Nature Physical Science 241, 20-22 (1973).
69. Zheng Y, Xiao M, Jiang S, Ding F, Wang J. Coating fabrics with gold nanorods for colouring, UV-protection, and antibacterial functions. Nanoscale 5, 788-795 (2013).
70. Ristaino J B, Groves C T, Parra G R. PCR amplification of the Irish potato famine pathogen from historic specimens. Nature 411, 695 (2001).

It will be understood that various details of the presently disclosed subject matter can be changed without departing from the scope of the presently disclosed subject matter. Furthermore, the foregoing description is for the purpose of illustration only, and not for the purpose of limitation.

1. A system for detecting a presence and/or an amount of one or more volatile organic compounds (VOC) in a plant sample, the system comprising:
a receiver configured to receive a gaseous emission from a plant sample, the receiver comprising one or more sensing elements, each sensing element comprising one or more sensors, that react with one or more VOC in the gaseous emission; and
a detector comprising one or more cameras, the detector being configured for detecting a signal associated with the reacting of the one or more sensing elements with one or more VOC in the gaseous emission.

2. The system of claim 1, wherein the one or more sensing elements comprise a nanosensor, a dye, or a combination thereof.

3. The system of claim 2, where the nanosensor is functionalized with a ligand that reacts with the one or more VOC in the gaseous emission.

4. The system of claim 2, wherein the nanosensor is shape-controlled, optionally wherein the nanosensor comprises a nanoparticle, a nanorod, or other shapes.

5. The system of claim 4, wherein the nanosensor has a dimension ranging from between, and including, about 5 and 200 nanometers (nm) and/or an aspect ratio of about 1 to about 6.

6. The system of claim 4, wherein the nanosensor comprises gold, silver, copper, aluminum, or any alloy thereof.

7. The system of claim 4, wherein the nanosensor, optionally the shape-controlled nanosensor, has an absorption range of between, and including, about 400 and 1200 nm, optionally wherein the nanosensor comprises a nanoparticle having an absorption range of between, and including, about 520 and 580 nm and/or wherein the nanosensor comprises a nanorod having a longitudinal resonance in the range of between, and including, about 530 and 1000 nm.

8. The system of claim 1, comprising between, and including, about 2 and 100 sensing elements, optionally, between, and including, about 2 and 10 sensing elements, further optionally wherein the sensing elements of the receiver are configured in a linear microarray.

9. The system of claim 1, wherein the one or more cameras of the detector is configured to capture one or more images of the receiver.

10. The system of claim 1, wherein the detector comprises a consumer electronics device comprising a light source configured to illuminate the receiver, optionally wherein the consumer electronics device is a smart phone, a tablet, or other mobile device.

11. The system of claim 9, wherein the detector comprises an attachment configured to position the receiver with respect to the camera and/or light source, optionally wherein the attachment further comprises a lens, a diffuser or a combination thereof.

12. The system of claim 1, comprising a pump configured to direct the gaseous emission to the receiver.

13. The system of claim 1, comprising one or more processors configured to determine the presence and/or the amount the one or more VOC in the plant sample.

14. The system of claim 1, wherein the receiver comprises a material selected from the group consisting of paper and a hydrophobic nanoporous substrate; and optionally, wherein the hydrophobic nanoporous substrate is selected from the group consisting of a silica sol-gel, a polymer membrane, and a metal organic framework (MOF).

15. A nanosensor that reacts with one or more VOC in a gaseous emission from a plant sample.

16. The nanosensor of claim 15, wherein the nanosensor is functionalized with a ligand that reacts with the one or more VOC in the gaseous emission.

17. The nanosensor of claim 14, wherein the nanosensor is shape-controlled, optionally wherein the nanosensor comprises a nanoparticle or a nanorod.

18. The nanosensor of claim 17, wherein the nanosensor has a dimension ranging between, and including, about 5 and 200 nanometers (nm) and/or an aspect ratio of between, and including, about 1 to 6.

19. The nanosensor of claim 17, wherein the nanosensor comprises gold, silver, copper, aluminum, or any alloy thereof.

20. The nanosensor of claim 15, wherein the nanosensor, optionally the shape-controlled nanosensor, has an absorption range of between, and including, about 400 and 1200 nm, optionally wherein the nanosensor comprises a nanoparticle having an absorption range of between, and including, about 520 and 580 nm, and/or wherein the nanosensor comprises a nanorod having a longitudinal resonance in the range of between, and including, about 530 and 1000 nm.

21. A method for detecting a presence and/or an amount of one or more volatile organic compounds (VOC) in a plant sample, the method comprising:
providing a plant sample;
exposing a gaseous emission from the plant sample to one or more sensing elements, each sensing element comprising one or more sensors, that react with the one or more VOC in the gaseous emission;
detecting a signal associated with the reacting of the one or more VOC with the one or more sensing elements; and
detecting the presence and/or the amount of the one or more VOC based on the signal.

22. The method of claim 21, wherein the one or more sensing elements comprise a nanosensor, a dye, or a combination thereof.

23. The method of claim 22, wherein the nanosensor is functionalized with a ligand that reacts with the one or more VOC in the gaseous emission.

24. The method of claim 22, wherein the nanosensor is shape-controlled, optionally wherein the nanosensor comprises a nanoparticle or a nanorod.

25. The method of claim 24, wherein the nanosensor has a dimension ranging between, and including, about 5 and 200 nanometers (nm) and/or an aspect ratio of between, and including, about 1 and 6.

26. The method of claim 24, wherein the nanosensor comprises gold, silver, copper, aluminum, or any alloy thereof.

27. The method of claim 24, wherein the nanosensor, optionally the shape-controlled nanosensor, has an absorption range of between, and including, about 400 and 1200 nm, optionally wherein the nanosensor comprises a nanoparticle having an absorption range of between, and including, about 520 and 580 nm and/or wherein the nanosensor comprises a nanorod having a longitudinal resonance in the range of between, and including, about 530 and 1000 nm.

28. The method of claim 21, wherein the one or more sensing elements are configured on a receiver, optionally wherein the receiver comprises a material selected from the group consisting of paper and a hydrophobic nanoporous substrate, further optionally wherein the hydrophobic nanoporous substrate is selected from the group consisting of a silica sol-gel, a polymer membrane, and a metal organic framework (MOF).

29. The method of claim 21, comprising between, and including, about 2 and 100 sensing elements, optionally between, and including, about 2 and 10 sensing elements, further optionally wherein the sensing elements of the receiver are configured in a linear microarray.

30. The method of 21, wherein detecting a signal comprises capturing one or more images of the one or more sensing elements with a camera.

31. The method of claim 21, wherein detecting a signal comprises using a consumer electronics device having a camera configured to capture one or more images of the receiver and a light source configured to illuminate the receiver, optionally wherein the consumer electronics device is a smart phone, a tablet, or some other mobile device.

32. The method of claim 31, wherein detecting a signal comprises employing an attachment configured to position the one or more sensing elements with respect to the camera and/or light source, optionally wherein the attachment further comprises a lens, a diffuser, or a combination thereof.

33. The method of claim 21, comprising directing the gaseous emission to the one or more sensing elements using a pump.

34. The method of claim 31, wherein the plant sample is a field sample or a sample from a plant product.

35. The method of claim 21, comprising determining a condition of the plant or plant product based on the presence and/or the amount of the one or more VOC.

36. The method of claim 35, wherein the condition of the plant is an infection, an asymptomatic infection, a contamination by a foodborne microorganism, an abiotic stress condition, a pest infestation, or a combination thereof.

37. The method of claim 36, wherein the infection is an infection caused by a fungus, bacterium, virus, oomycete, other plant pathogen, or insect pest.

38. The method of claim 35, comprising generating a profile of one or more signals from the one or more sensing elements based on the condition of the plant.

39. The method of claim 35, wherein generating a profile comprises generating a profile to identify and/or distinguish individual species of organism.

40. A profile generated by the method of claim 38.

41. A profile generated by the method of claim 39.
